(19) United States
(12) Patent Application Publication
(10) Pub. No.: US 2006/0115448 A1
(43) Pub. Date: Jun. 1, 2006

(54) Amphiphilic polynorbornene derivatives and methods of using the same

(76) Inventors: Gregory N. Tew, Amherst, MA (US); M. Firat Ilker, Madison, WI (US); E. Bryan Coughlin, Amherst, MA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/206,378

(22) Filed: Aug. 18, 2005

Related U.S. Application Data
(60) Provisional application No. 60/602,362, filed on Aug. 18, 2004.

(51) Int. Cl. A61K 31/78; A61K 31/785; C08F 26/08

(52) U.S. Cl. 424/78.3; 526/264

(57) ABSTRACT

Polynorbornene derivatives exhibiting antibacterial activity and low hemolytic activity are described herein. Antimicrobial compositions and pharmaceutical compositions comprising polynorbonene derivatives and methods of using the same are also described. Such compositions, which exhibit substantial antibacterial activity and low hemolytic activity, may be suitable for material applications and therapeutic uses.

### CROSS REFERENCE TO RELATED APPLICATIONS

This application claims priority to U.S. Provisional Application No. 60/602,362 entitled “Non-Hemolytic Amphiphilic Cationic Polymers via ROMP” filed on Aug. 18, 2004, the entire contents of which is incorporated herein by reference.

### BACKGROUND

Antibacterial activities of macromolecules, including oligomeric compounds, have been studied under two major areas, for the most part independent from each other. One group of studies has focused on the structure-property relationships of natural host-defense peptides derived from multicellular organisms. These peptides have a great diversity with regard to their length, amino acid composition and antimicrobial activities ranging from very potent to weak. Despite this diversity, most are cationic peptides with a certain degree of hydrophobicity. Extensive studies on the mechanism of action suggest that antimicrobial peptides act by permeabilizing the cell membranes of microorganisms through favorable interactions with negatively charged and hydrophobic components of the membranes followed by aggregation and subsequent disruption. This mechanism is suggested to be responsible for the wide spectrum of potency and speed of action for these antibacterial peptides. Host-defense peptides and their synthetic analogs are reported to exhibit varying degrees of activity against different bacteria and mammalian cells. While host-defense peptides may show selectivity against the membranes of microbes versus the host organism, a number of them are antibacterial and not toxic to human cells, within certain concentration limits, and are thus considered as potential therapeutic agents. The selective action has been suggested to be due to the balance and spatial arrangement of hydrophobic and hydrophilic components of the peptide that distinguishes between the more negatively charged outer surface of microbial membranes and the neutral and cholesterol rich membranes of multicellular animals. Studies aimed at understanding the structure-property relationships of natural peptides have recently evolved into a number of research efforts targeting the preparation of synthetic mimics of antimicrobial peptides. These include stereoisomers of natural peptides, α-peptides, β-peptides, cyclic α-peptides, peptoids, and polyarylamides, all of which are oligomeric with molecular weight below 3000 g/mol. Many of these examples target an amphiphilic secondary structure, typically helical, in addition to their cationic nature. Depending on the type of peptide, a facially amphiphilic structure results in the gain, or loss, of selective activity, which reveals that a stable amphiphilic secondary structure is not a precondition for selective antibacterial activity. Resistance to enzymatic degradation was also targeted in some cases for potential use in therapeutic applications.

Independent from the antimicrobial peptide research, a second area involves studies of synthetic cationic polymers that exhibit varying degrees of antibacterial activities. This class of polymeric compounds is relatively inexpensive and less cumbersome to prepare, when compared to peptide mimics. In many instances, cationic polymers were reported to exhibit enhanced antibacterial activities compared to their small molecule counterparts. The most common polymers are quaternary ammonium, poly quats, and phosphonium functionalized polymers. This class of polymers was predominantly targeted for use in the solid state as potent disinfectants, biocidal coatings or filters, due to their toxicity to human cells at relatively low concentrations which is an important distinction from the work on peptide mimics. Consistent with the target applications of these cationic polymers, in most cases only antibacterial activity was reported without any report of hemolytic activity. In one instance, a soluble pyridinium polymer was reported to have low acute toxicity against the skin of test animals. Two examples of antibacterial cationic polymers that have found large industrial use as disinfectants and biocides are poly(hexamethylene biguanide)s (PHMB) and poly-ε-lysine. Different levels of toxicity against various mammalian cells were reported for PHMB and similar biguanide functionalized polymers. Poly-ε-lysine is considered to be an environmentally friendly antimicrobial preservative in most part due to its biodegradability into non-toxic components. A direct comparison of antibacterial and hemolytic action has not been reported for either of these classes of antimicrobial polymers. Gelman et al. has recently reported the antibacterial activity of low molecular weight, hydrophobically modified, cationic polystyrenes in comparison with a potent derivative of magainin II. In their initial study, a crossover between the research on antimicrobial peptide mimics and polymer disinfectants, cationic polystyrenes has shown similar antibacterial activities as the magainin derivative, but were highly hemolytic. Recently, selective activities of facially amphiphilic low molecular weight polyphenyleneethynylenes with activity and selectivity similar to a magainin derivative was reported. The successful design of non-hemolytic, antibacterial, and high molecular weight polymers by tuning their membrane disruption activities has remained unanswered thus far. Ring-opening metathesis polymerization (ROMP) has been successfully used in the preparation of biologically active well-defined polymeric materials, due to its living nature and functional group tolerance. Examples included polymers carrying oligopeptides, oligonucleotides, carbohydrates, anti-cancer drugs, and antibiotic agents. ROMP-based techniques are evolving into a powerful synthetic toolbox for the introduction of multiple functionalities into polymeric materials in pursuit of obtaining potent biological activities. The synthesis and ROMP of modular norbornene derivatives for the preparation of well-defined amphiphilic polymers exhibiting lipid membrane disruption activities was reported. Cationic amphiphilic polymers above certain molecular weights appeared to show the highest membrane disruption activities on lipid vesicles as rough models for bacterial membranes.

The antibacterial and hemolytic activities of narrow polydispersity homopolymers and random copolymers of modular norbornene derivatives, spanning a large range of molecular weights are presented herein. Results indicate that by controlling the hydrophobic/hydrophilic balance of water soluble amphiphilic polymers, it is possible to obtain high selectivity between antibacterial and hemolytic activities without a predisposed amphiphilic secondary structure as part of the synthetic design. The overall efficacy toward both Gram-negative and Gram-positive bacteria appears to be dependent on the length of alkyl substituents on the repeat units. Therefore, it is possible to design simple polymers that are both potent against bacteria and non-hemolytic.

### SUMMARY

One embodiment of the present invention provides polymers and methods of their use, including the use of polymers as antimicrobial agents in pharmaceutical and non-pharmaceutical applications. A further embodiment of the present invention provides compositions of the polymers and methods of preparing the polymers.

One embodiment of the present invention is an polymer comprising a first polynorbornene monomer and a second polynorbornene monomer. In some embodiments, the first and second polynorbornene monomers may be different or the same. In further embodiments, the monomers may be such that the polymer exhibits a random, block or alternating pattern. In certain embodiments, the polymer may comprise monomer units with a hydrophilic and a hydrophobic side chain or face, such that the monomer unit is amphiphilic. In another embodiment, the polymer may comprise monomer units with a hydrophilic side chain and monomer units with a hydrophobic side chain, the two types of monomers being distributed along the polymer backbone.

Another embodiment is an amphiphilic monomer comprising a polynorbornene of the formula: 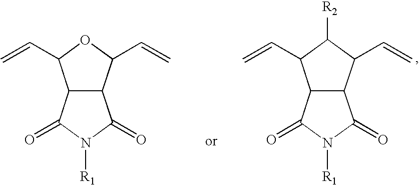
wherein R₁ may be polar or non-polar and R₂, if present, is of the opposite polarity of R₁. In further embodiments, an amphiphilic polymer formed from the polynorbornene monomeric units is provided, such that the polymer is amphiphilic. The polymer may be a homopolymer or a copolymer.

In a preferred embodiment, the polynorbornene is selected from the group consisting of: 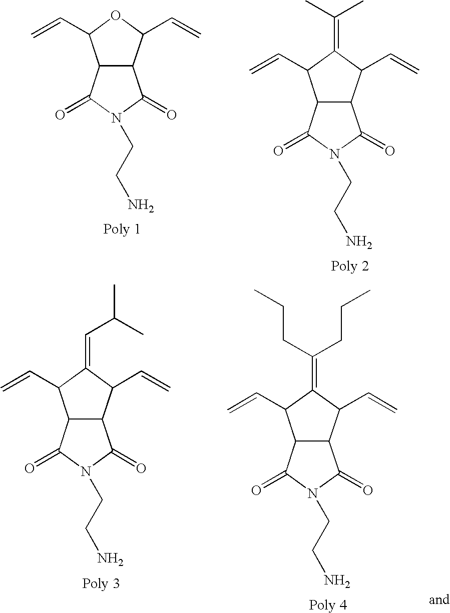
combinations thereof. In more preferred embodiments, an amphiphilic polymer comprising poly3 is provided. In another more preferred embodiment, an amphiphilic copolymer comprising poly2 and poly3 is provided. In such amphiphilic copolymers, the monomeric units may be distributed in block, random or alternating units along the backbone.

A further embodiment is an amphiphilic copolymer comprising a polar polynorbornene monomeric unit and a non-polar polynorbornene monomeric unit. In preferred embodiments, the polynorbornene monomeric units may be selected from the group consisting of the following formulas: 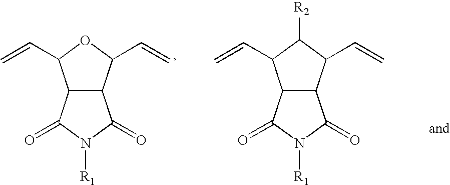
combinations thereof, wherein R₁ may be polar or non-polar and R₂, if present, may polar or non-polar, such that the monomers may be hydrophilic or hydrophobic. In such amphiphilic copolymers, the monomeric units may be distributed in block, random or alternating units along the backbone.

Another embodiment is a pharmaceutical composition comprising an amphiphilic polymer or copolymer comprising polynorbornene monomers and a pharmaceutically acceptable excipient or diluent. In one embodiment, the amphiphilic polymer of the pharmaceutical composition may comprise a homopolymer of amphiphilic polynorbornene monomers or a copolymer of amphiphilic polynorbornene monomers. In another embodiment the amphiphilic copolymer of the pharmaceutical composition may comprise a polar polynorbornene monomer and a non-polar polynorbornene monomer. In further embodiments, the monomers may be present in the copolymers such that the polymer exhibits a random, block or alternating pattern.

Another embodiment of the present invention is a method of treating microbial or bacterial infections comprising administering a therapeutically effective amount of an amphiphilic polymer or copolymer as described herein or a pharmaceutical composition containing the same.

A further embodiment of the present invention is directed to a method of providing an antidote to low molecular weight heparin overdose comprising administering an amphiphilic polymer or copolymer as described herein.

Another embodiment is directed to a method of inhibiting or preventing the growth of a microorganism, the method comprising contacting the microorganism with an effective amount of an amphiphilic polynorbornene polymer or copolymer. In further embodiments, the polymer or copolymer may be attached to or present on a substrate.

A further aspect of the present invention provides an antimicrobial composition comprising a polynorbornene polymer or copolymer as described herein and a composition selected from the group consisting of paints, lacquers, coatings, varnishes, caulks, grouts, adhesives, resins, films, cosmetics, soaps, lotions, handwashes, and detergents.

A further embodiment of the present invention is directed to coatings comprising a polynorbornene polymer or copolymer. Such coatings may be useful for various material applications, including HVAC systems, electronic components and the like.

### DESCRIPTION OF DRAWINGS

For a fuller understanding of the nature and advantages of the present invention, reference should be had to the following detailed description taken in connection with the accompanying drawings, in which:

FIG. 1. Hemolysis curves of poly2 (A) and poly3 (B) at increasing concentrations.

FIG. 2. Lysis of neutral vesicles (Cholesterol/SOPC) and negatively charged vesicles (SOPS/SOPC), at 3 minutes, caused by 25 μg/mL of poly2 (A), Mn=10050 g/mol, poly(2₂-co-3₁) (B), Mₙ=15300 g/mol, and poly(3) (C), Mₙ=10300 g/mol. Percent lysis values are given on top of the bars.

FIG. 3. Colony count of polyurethane paint untreated and treated with 0.5% weight and 1.0% weight poly3.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to an “fibroblast” is a reference to one or more fibroblasts and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

The methods as described herein for use contemplate prophylactic use as well as curative use in therapy of an existing condition. As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a copolymer, can include, but is not limited to, providing a copolymer systemically to a patient by, e.g., intravenous injection whereby the therapeutic reaches the target tissue; oral ingestion, whereby the therapeutic reaches the target tissue. “Administering” a composition may be accomplished by injection, topical or oral administration, or by any method in combination with other known techniques.

As used herein, the term “therapeutic” means an agent utilized to treat, combat, ameliorate, prevent or improve an unwanted condition or disease of a patient. In part, embodiments of the present invention are directed to decrease or prevent bacterial infection in a patient.

A “therapeutically effective amount” or “effective amount” of a composition is a predetermined amount calculated to achieve the desired effect, i.e., to treat or prevent bacterial infection. A therapeutically effective amount of a copolymer of the present invention is typically an amount such that when it is administered in a physiologically tolerable excipient composition, it is sufficient to achieve an effective systemic or local concentration in the tissue. Effective amounts of compounds of the present invention can be measured by improvements in patient symptoms or microbial count or concentration and the like.

One embodiment of the present invention provides non-peptidic, amphiphilic monomers and polymers and random copolymers of such monomers and methods of using in a number of applications, including their use in pharmaceutical and non-pharmaceutical applications as antimicrobial agents. A further embodiment of the present invention provides compositions comprising such amphiphilic polynorbornene monomers, polymers and copolymers and methods for preparing the same.

The monomers of the present invention are polynorbornenes of the formula: 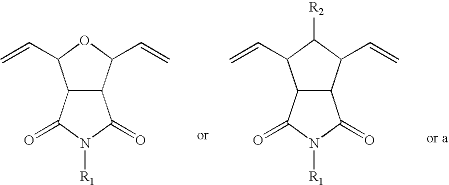
combination thereof, wherein R₁ is polar or non-polar and R₂, if present, is polar or non-polar, such that the monomers are amphiphilic. In preferred embodiments, the monomers may be selected from the group consisting of: 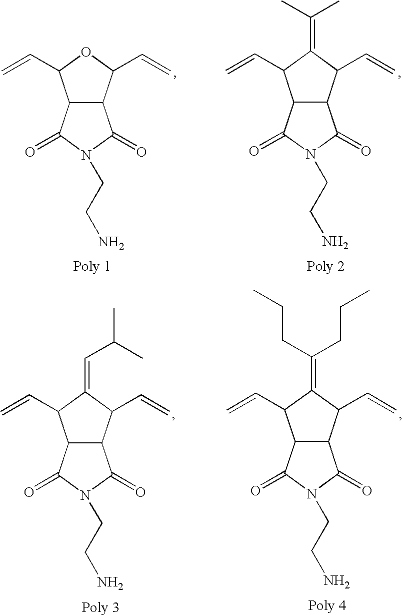
and combinations thereof. Such amphiphilic polynorbornene monomers may be polymerized to form polymers or copolymers. In a preferred embodiment, an amphiphilic polymer comprises poly3. In another preferred embodiment, an amphiphilic copolymer comprises poly2 and poly3, preferably in a ratio of about 10:1 to about 1:10, more preferably about 1:1 and in a random pattern.

Another embodiment is an amphiphilic copolymer comprising a polar polynorbornene monomeric unit and a non-polar polynorbornene monomeric unit. The ratio of polar to non-polar monomers within a copolymer may range from about 100:1 to about 1:100, preferably 10:1 to about 1:10, more preferably about 1:1. In preferred embodiments, the monomeric units may include

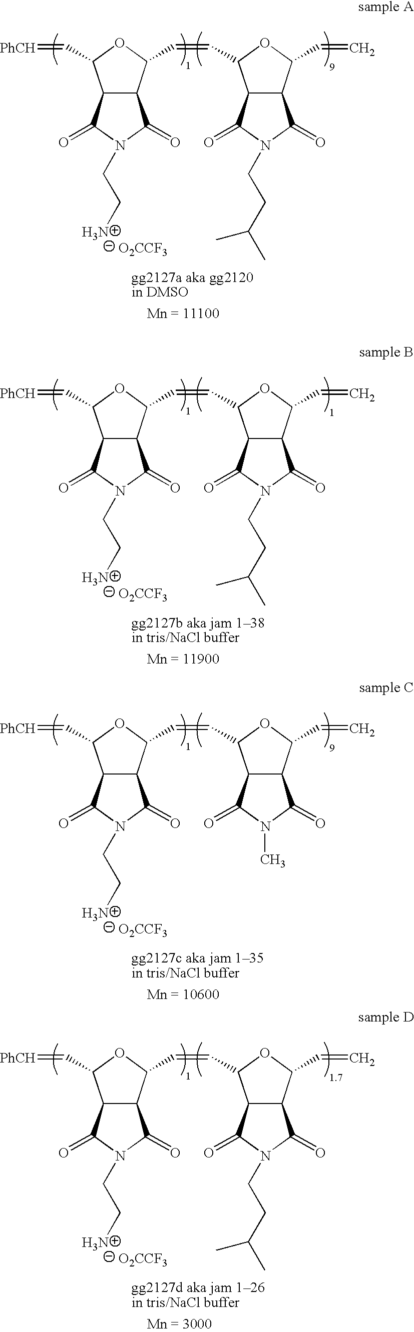

Examples of polar and non-polar groups or side chains of the polynorbornene monomeric units of the present invention include alkyls, alkylenes, alkylynes, aryls, arylenes, alkoxy, cycloalkyls, halogens, heteroaryls, heterocycles, alkylaminos, and alkylthio groups. In preferred embodiments, the polar group or side chain may be (CH₂CH₂NH)n—CH₂CH₂NH₂, wherein n=1, 2 or 3; 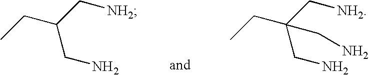
More preferred polar groups include methylamine, ethylamine and butylamine. Preferred non-polar groups include methyl, ethyl, propyl, butyl, isobutyl and pentyl.

In further embodiments, dendritic derivatives of R₁ may be synthesized, for example, R₁ may be

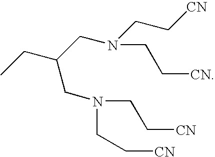

The polymers of the present invention may be homopolymers of amphiphilic norobornene monomers or random copolymers composed of monomer units with hydrophilic and hydrophobic side chains. Such monomer units may be randomly distributed along the copolymer backbone.

A further embodiment of the present invention provides methods of preparing such polymers and copolymers. In one embodiment, the polymers may be prepared by copolymerization of monomer unit precursors. In a further embodiment, random copolymers may be synthesized by copolymerization of different monomer precursors. The desired comonomer content and molecular weight may be controlled by altering the comonomer feed ratio and catalyst to monomer ratio.

The random copolymers of the invention can be synthesized using a chain transfer agent to control the degree of polymerization and, accordingly, have average degrees of polymerization and average molecular weights that are lower than those of copolymers synthesized without a chain transfer agent. Copolymers of the present invention typically have average degrees of polymerization of about four (4) or five (5) to about 50 to 100. Preferred copolymers have average degrees of polymerization ranging from about 4 or 5 to about 20, or from about 5 to about 30.

Use of a chain transfer agent to control the degree of polymerization results in the preparation of the low molecular weight copolymers of the present invention at relatively high yields and avoids the necessity of time-intensive fractionation by column chromatography, which is usually required to obtain low molecular weight polymers in polymerizations performed without a chain transfer agent. The copolymers of the present invention are thus easy to prepare, inexpensive, and suitable for industrial-scale production.

The polymers and copolymers of the present invention are amphiphilic and capable of disrupting the integrity of the cell membrane of microorganisms, which results in the inhibition of growth or the death of the microorganisms. As a consequence, the polymers and copolymers possess antimicrobial activity, including antibacterial, antifungal, and antiviral activity, and are useful as antimicrobial agents. The polymers and copolymers of the invention have a broad range of antimicrobial activity and are effective against a variety of microorganisms, including gram-positive and gram-negative bacterial, fungi, yeast, mycoplasmas, mycobacteria, protozoa, and the like. Moreover, through selection of the molecular weight and/or the hydrophobic side chain, the relative antimicrobial and hemolytic properties of the polymers and copolymers of the present invention can be controlled to produce antimicrobial polymers and copolymers that are non-toxic to mammals.

The polymers and copolymers of the present invention are useful as antimicrobial agents in a number of applications. For example, the polymers of the present invention can be used therapeutically to treat microbial infections in animals, including humans and non-human vertebrates such as wild, domestic and farm animals. The microbial infection in an animal is treated by administering to the animal an effective amount of a pharmaceutical composition of a polymer or copolymer of the present invention. The copolymer compositions can be administered systemically or topically and can be administered to any body site or tissue. Because the polymers and copolymers have a broad range of antimicrobial activity, they are useful in treating a variety of infections in an animal.

The amphiphilicity of the polymers and copolymers of the present invention form the basis for another therapeutic use, as antidotes for hemorrhagic complications associated with heparin therapy. Thus, the polymers and copolymers of the present invention can be used in a method of providing an antidote to heparin overdose in an animal by administering to the animal an effective amount of a pharmaceutical composition of the polymer or copolymer.

The polymers and copolymers of the present invention also can be used as disinfectants or as preservatives. The polymers and copolymers of the present invention can thus be used in a method of killing or inhibiting the growth of a microorganism by contacting the microorganism with an effective amount of the polymer or copolymer. For example, the copolymers of the present invention can be used as disinfectants or preservatives in, for example, cosmetics, soaps, lotions, handwashes, paints, cleansers, and polishers, and the like, or in, for example, foodstuffs, food containers, and food-handling implements. The copolymers are administered for these purposes as a solution, dispersion, or suspension. The polymers and copolymers of the present invention can also be incorporated into plastics that can be molded or shaped into articles, or attached or immobilized on a surface, to provide a surface-mediated microbicide that kills or inhibits the growth of microorganisms in contact with the surface. Moreover, by selecting the molecular weight and/or hydrophobic group of the polymers and copolymers of the present invention, the physical properties can be optimized for specific applications. For example, copolymers of the invention having long alkyl chains may be glassier due to the higher melting points of the long-chain alkyl groups and thus better suited for use in certain applications. Water-soluble amphiphilic polymers (for example cellulose derivatives) have been utilized as thickeners in foods or paints. Viscosity of the polymer solutions may be controlled by altering the molecular weight and compositions of the hydrophobic groups.

The present invention discloses amphiphilic polymers and copolymers. Polymers are generally defined as synthetic compounds assembled from monomer subunits and are polydisperse in molecular weight Polymers are most commonly prepared by one-pot synthetic procedures. The term “polymer,” as used herein, refers to a macromolecule comprising a plurality of repeating monomers or monomer units. The term “polymer” can include homopolymers, which are formed from a single type of monomer, and copolymers, which are formed from two or more different monomers. The term “copolymer” includes polymers in which the monomers are distributed randomly (random copolymer), in alternating fashion (alternating copolymers), or in blocks (block copolymer). The copolymers of the present invention are random copolymers. The term “random copolymer,” as used herein, refers to copolymers in which the monomers are distributed randomly.

The polymers and copolymers may have monomer units of the formula 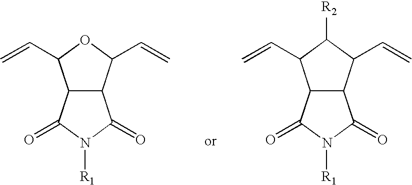
or combinations thereof wherein, R₁ is polar or non-polar and R₂ is non-polar, such that the monomers may be hydrophilic, hydrophobic or amphiphilic. In one preferred embodiment, the monomers may be amphiphilic and a polymer or copolymer comprising such amphiphilic monomer units may be formed. In another preferred embodiment, the monomers may be hydrophobic and hydrophilic and an amphiphilic copolymer comprising such hydrophilic and hydrophobic monomers may be formed.

Preferred polymers and copolymers of the present invention are also those wherein the average degree of polymerization (“DP”) is about 4 to about 50, about 4 to about 30, about 5 to about 25, about 5 to about 20, about 5 to about 15, about 5 to about 10, about 5 to about 12, about 5 to about 10, or about 6 to about 8. In some aspects of the invention, preferred polymers and copolymers are those wherein the DP is about 4 to about 15, or about 4 to about 10. Especially preferred are those copolymers wherein DP is about 4 to about 10, or about 6 to about 8.

In some embodiments of the present invention, preferred polymers and copolymers are those wherein DP is about 5 to about 50, about 5 to about 30, about 5 to about 20, about 6 to about 20, about 6 to about 15, about 6 to about 12, about 6 to about 10, or about 6 to about 8. Especially preferred are those wherein DP is about 6 to about 10, or about 6 to about 8.

Preferred polymers and copolymers of the present invention are those wherein n is 1-m, and m is about 0.1 to about 0.9, about 0.1 to about 0.6, about 0.35 to about 0.60, about 0.35 to about 0.55, about 0.50 to about 0.60, about 0.45 to about 0.55, or about 0.35 to about 0.45.

The polymers and copolymers of the present invention have about 4 monomer units to about 50 to 100 monomer units, with average molecular weights that range from about 500 Daltons to about 10,000 to 20,000 Daltons, or about 1,000 Daltons to about 10,000 to 20,000 Daltons. Preferred copolymers are those having about 4 to about 30 monomer units, about 5 to about 30 monomer units, about 4 to about 20 monomer units, or about 5 to about 20 monomer units, with average molecular weights that range from about 500 Daltons to about 10,000 Daltons, about 1,000 Daltons to about 10,000 Daltons, about 1,000 Daltons to about 5,000 Daltons, or about 1,000 Daltons to about 4,000 Daltons. Especially preferred polymers and copolymers are those having about 5 to about 10 monomer units, or about 6 to about 8 monomer units, with average molecular weights that range from about 500 Daltons to about 2,000 Daltons, or about 1,000 Daltons to about 2,000 Daltons.

The term “polymer backbone,” “copolymer backbone” or “backbone” as used herein refers to that portion of the polymer which is a continuous chain comprising the bonds formed between monomers upon polymerization. The composition of the polymer backbone can be described in terms of the identity of the monomers from which it is formed without regard to the composition of branches, or side chains, of the polymer backbone.

The term “polymer side chain”, “copolymer side chain” or “side chain” refers to portions of the monomer which, following polymerization, forms an extension of the polymer backbone.

The term “amphiphilic” as used herein describes a structure having discrete hydrophobic and hydrophilic regions. An amphiphilic polymer or copolymer requires the presence of both hydrophobic and hydrophilic elements along the backbone.

The term “microorganism” as used herein includes bacteria, algae, fungi, yeast, mycoplasmas, mycobacteria, parasites and protozoa.

The term “antimicrobial,” “microbiocidal,” or “biocidal” as used herein means that the materials inhibit, prevent, or destroy the growth or proliferation of microorganisms. This activity can be either bacteriocidal or bacteriostatic. The term “bactoriocidal” as used herein means the killing of microorganisms. The term “bacteriostatic” as used herein refers to inhibiting the growth of microorganisms which can be reversible under certain conditions.

The term “alkyl” as used herein by itself or as part of another group refers to both straight and branched-chain aliphatic hydrocarbon radicals from 1 to 12 carbons, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl.

The term “alkylene” as used herein refers to straight chain or branched divalent aliphatic hydrocarbon radicals from 1 to 20 carbon atoms in length, or, more preferably, from 1 to 10 carbon atoms, or from 1 to 6 carbon atoms in length. Examples of alkylene radicals include, but are not limited to, methylene (—CH₂—), ethylene (—CH₂CH₂—), propylene isomers (e.g., —CH₂CH₂CH₂— and —CH(CH₃)CH₂—), and the like.

The term “alkoxy” as used herein refers to a straight or branched chain aliphatic hydrocarbon radicals of 1 to 20 carbon atoms, unless the chain length is limited thereto, bonded to an oxygen atom, including, but not limited to, methoxy, ethoxy, n-propoxy, isopropoxy, and the like. Preferably, the alkoxy chain is 1 to 10 carbon atoms in length, more preferably 1 to 8 carbon atoms in length, and even more preferred 1 to 6 carbon atoms in length.

The term “aryl” as used herein by itself or as part of another group refers to monocyclic or bicyclic aromatic groups containing from 6 to 12 carbons in the ring portion, preferably 6-10 carbons in the ring portion, such as the carbocyclic groups phenyl, naphthyl and tetrahydronaphthyl.

The term “arylene” as used herein refers to divalent aryl groups (e.g., monocyclic or bicyclic aromatic groups) containing from 6 to 12 carbons in the ring portion, preferably 6-10 carbons in the ring portion, that are derived from removal of a hydrogen atom from two ring carbon atoms. Examples of arylene groups include, but are not limited to o-phenylene, naphthylene, benzene-1,2-diyl and the like.

The term “cycloalkyl” as used herein by itself or as part of another group refers to cycloalkyl groups containing 3 to 9 carbon atoms, more preferably, 3 to 8 carbon atoms. Typical examples are cyclopropyl, cyclobutyl, cyclopentyl, cyclohexyl, cycloheptyl, cyclooctyl and cyclononyl.

The term “halogen” or “halo” as used herein by itself or as part of another group refers to chlorine, bromine, fluorine or iodine.

The term “heteroaryl” as used herein refers to groups having 5 to 14 ring atoms; 6, 10 or 14 7π-electrons shared in a cyclic array; and containing carbon atoms and 1, 2 or 3 oxygen, nitrogen or sulfur heteroatoms. Examples of heteroaryl groups include thienyl, imadizolyl, oxadiazolyl, isoxazolyl, triazolyl, pyridyl, pyrimidinyl, pyridazinyl, furyl, pyranyl, thianthrenyl, pyrazolyl, pyrazinyl, indolizinyl, isoindolyl, isobenzofuranyl, benzoxazolyl, xanthenyl, 2H-pyrrolyl, pyrrolyl, 3H-indolyl, indolyl, indazolyl, purinyl, 4H-quinolizinyl, isoquinolyl, quinolyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, and phenoxazinyl groups. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine. The term “heteroarylene” as used herein refers to divalent heteroaryl groups that are derived from removal of a hydrogen atom from two ring atoms.

The term “heterocycle,” “heterocyclic,” or “heterocyclic ring”, as used herein except where noted, represents a stable 5- to 7-membered mono- or bicyclic or stable 7- to 10-membered bicyclic heterocyclic ring system any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Especially useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

The term “alkylamino” as used herein by itself or as part of another group refers to an amino group which is substituted with one alkyl group having from 1 to 6 carbon atoms. The term “dialkylamino” as used herein by itself or as part of an other group refers to an amino group which is substituted with two alkyl groups, each having from 1 to 6 carbon atoms.

The term “alkylthio” as used herein by itself or as part of an other group refers to an thio group which is substituted with one alkyl group having from 1 to 10 carbon atoms, or, preferably, from 1 to 6 carbon atoms.

Generally and unless defined otherwise, the phrase “optionally substituted” used herein refers to a group or groups being optionally substituted with one or more substituents independently selected from the group consisting of amino, hydroxy, nitro, halogen, cyano, thiol, C₁₋₆ alkyl, C₂₋₆ alkenyl, C₂₋₆ alkynyl, C₃₋₆ cycloalkyl, and C₁₋₆ aryl.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “animal” as used herein includes, but is not limited to, humans and non-human vertebrates such as wild, domestic and farm animals.

In some aspects of the invention, the polymers and copolymers of the present invention are derivatives referred to as prodrugs. The expression “prodrug” denotes a derivative of a known direct acting drug, which derivative has enhanced delivery characteristics and therapeutic value as compared to the drug, and is transformed into the active drug by an enzymatic or chemical process.

When any variable occurs more than one time in any constituent or in any of the copolymers recited for any of the formulae above, its definition on each occurrence is independent of its definition at every other occurrence. Also, combinations of substituents and/or variables are permissible only if such combinations result in stable compounds.

It is understood that the present invention encompasses the use of stereoisomers, diastereomers and optical isomers of the polymers and copolymers of the present invention, as well as mixtures thereof, for treating microbial infections, killing or inhibiting the growth of a microorganism, and providing an antidote to low molecular weight heparin overdose in an animal. Additionally, it is understood that stereoisomers, diastereomers and optical isomers of the polymers and copolymers of the present invention, and mixtures thereof, are within the scope of the invention. By way of non-limiting example, the mixture may be a racemate or the mixture may comprise unequal proportions of one particular stereoisomer over the other. Additionally, the polymers and copolymers of the present invention may be provided as a substantially pure stereoisomers, diastereomers and optical isomers.

In another aspect of the invention, the polymers and copolymers of the present invention, in particular, those with cationic side chains, can be provided in the form of an acceptable salt (i.e., a pharmaceutically acceptable salt) for treating microbial infections, killing or inhibiting the growth of a microorganism, and providing an antidote to low molecular weight heparin overdose in an animal. Polymer and copolymer salts can be provided for pharmaceutical use, or as an intermediate in preparing the pharmaceutically desired form of the copolymer. One copolymer salt that can be considered to be acceptable is the hydrochloride acid addition salt. For example, chloride ion can be present as a counter ion for polymers and copolymers having cationic side chains. Hydrochloride acid addition salts are often acceptable salts when the pharmaceutically active agent has an amine group that can be protonated. Since a polymer or copolymer of the invention may be polyionic, such as a polyamine, the acceptable copolymer salt may be provided in the form of a poly(amine hydrochloride). Other acceptable salts include conjugate bases of pharmaceutically acceptable acids, such as, for example, trifluoroacetate, the conjugate base of the pharmaceutically acceptable acid trifluoroacetic acid (TFA).

The polymers and copolymers of the present invention have been shown to possess antimicrobial activity. Thus, the polymers and copolymers of the present invention can be used as antimicrobial agents and, for example, can be used in a method of treating microbial infections in an animal.

Thus, the invention is directed to a method of treating a microbial infection in an animal in need thereof, by administering to the animal a polymer or copolymer of the present invention.

For example, in some aspects, the invention is directed to a method of treating a microbial infection in an animal in need thereof, the method comprising administering to the animal an effective amount of a pharmaceutical composition comprising a polymers or copolymer, as defined above, and a pharmaceutically acceptable carrier or diluent, or an effective amount of a pharmaceutical composition comprising a polymer or copolymer as defined above.

The polymers and copolymers of the present invention can be used to treat a microbial infection caused by any type of microorganism, including, but not limited to, bacteria, algae, fungi, yeast, mycoplasmas, mycobacterial, parasites and protozoa. The copolymers of the present invention are therefore effective in treating bacterial infections, fungal infections, viral infections, yeast infections, mycoplasmid infections, mycobacterial infections, or protozoal infections.

The polymers and copolymers of the present invention have also been shown to possess antiviral activity and can be used as antiviral agents.

Thus, in some aspects, the invention is directed to a method of treating a viral infection in an animal in need thereof, the method comprising administering to the animal an effective amount of a pharmaceutical composition comprising a polymer or copolymer, as defined above, and a pharmaceutically acceptable carrier or diluent.

The polymers and copolymers of the present invention can also be used in methods of treating fungal infections.

Immunocompromised individuals are at serious risk for developing systemic fungal infections and the high incidence of cancer and AIDS underscores the need for developing effective and safe antifungal therapies. Many of the existing antifungal drugs act on molecular targets involved in cell wall synthesis (Debono, M., and Gordee, R. S., Ann. Rev. Microbiol. 48:471-497 (1994)). However, many of these targets are also found in mammalian cells which can lead to unwanted side-effects, and current therapies are associated with serious clinical complications including hepatic and kidney toxicities. Furthermore, as with bacterial infections, drug-resistant fungi are emerging at an alarming rate (DeLucca, A. J., and Walsh, T. J., Antimicob. Agents Chemother. 43:1-11 (1999)). Therefore, there is a strong need for the development of novel approaches for systemic and topical agents that can rapidly, effectively and safely control fungal infections while minimizing the potential for the development of resistance to their mechanism of action.

The polymers and copolymers of the present invention have also been shown to possess antifungal activity and thus can be used as antifungal agents, for example, in a method of treating fungal infections in an animal.

Thus, in some aspects, the invention is directed to a method of treating a fungal infection in an animal in need thereof, the method comprising administering to the animal an effective amount of a pharmaceutical composition comprising a polymer or copolymer, as defined above, and a pharmaceutically acceptable carrier or diluent.

The polymers and copolymers of the invention can also be used as antidotes for hemorrhagic complications associated with low molecular weight heparin therapy.

Heparin has been commonly used as an anticoagulant and antithrombotic agent in the hospital setting. However, there are several pharmacokinetic parameters of standard heparin (SH) that complicate therapy. For example, the high serum protein-binding activity of SH precludes subcutaneous administration and its rapid and unpredictable plasma clearance necessitates constant monitoring of activated partial thromboplastin time to assess effectiveness (Turpie, A. G. G., Am. Heart J. 135:S329-S335 (1998)). More recently, low molecular weight heparin derivatives (LMWH) have become the standard of care for the management of major vessel thrombotic conditions (Hirsh, J., and Levine, M. N., Blood. 79:1-17 (1992)). Nevertheless, LMWHs have gained popularity over standard heparin (SH) as antithrombotic agents because of their improved pharmacokinetics and more predictable anticoagulant responses to weight-adjusted doses. LMWHs are formed by enzymatic or chemical cleavage of heparin and are effective factor Xa inhibitors because they contain the high affinity pentasaccharide sequence. However, they are not effective thrombin inhibitors (Hirsh, J., and Levine, M. N., Blood. 79:1-17 (1992)).

Both SH and LMWH have a high net negative (anionic) charge. Hemorrhagic complications are associated with antithrombotic treatments with both agents and an overdose may result in serious bleeding. Protamine, by virtue of its positive charge, can neutralize the effects of the heparin but protamine therapy also has serious adverse, side-effects including hypotension, pulmonary hypertension and impairment of certain blood cells including platelets and lymphocytes (Wakefield, T. W., et al., J. Surg. Res. 63:280-286 (1996)). Therefore, there is a strong need for the development of safe and effective antidotes for hemorrhagic complications associated with SH and LMWH antithrombotic therapies.

The polymers and copolymers of the present invention have been shown to inhibit the anticoagulation effects of heparin, in particular, low molecular weight heparin, and can be used as antidotes for hemorrhagic complications associated with low molecular weight heparin therapy.

Thus, in some aspects, the invention is directed to a method of providing an antidote to low molecular weight heparin overdose in an animal in need thereof, the method comprising administering to the animal an effective amount of a pharmaceutical composition comprising a polymer or copolymer, as defined above, and a pharmaceutically acceptable carrier or diluent, or an effective amount of a pharmaceutical composition comprising a polymer or copolymer having a monomer unit as defined above.

In further aspects of the invention, the polymers and copolymers of the present are useful as therapeutic agents. In one particular embodiment, the polymers may be useful in oral or periodontal applications for treating or preventing oral diseases or disorders. Exemplary delivery methods include, but are not limited to, oral administration, such as a mouthwash, gum, toothpaste, liquid, foam and gel, parenteral administration or incorporation into an implantable device for controlled and/or sustained release of the agent.

In some aspects of the invention, the polymers and copolymers of the present invention are useful as disinfectants. For example, coatings and paints adhesives are all exposed to microbial contamination and are used in locations where microbial growth is undesirable. Thus, the copolymers of the present invention are incorporated into polishes, paints, sprays, or detergents formulated for application to surfaces to inhibit the growth of a bacterial species thereon. These surfaces include, but are not limited to surfaces, such as, countertops, desks, chairs, laboratory benches, tables, floors, bed stands, tools or equipment, doorknobs, windows, and drywall. Copolymers and polymers of the present invention are also incorporated into soaps, cosmetics, lotions, such as hand lotions, and handwashes. The present cleansers, polishes, paints, sprays, soaps, cosmetics, lotions, handwashes, or detergents contain polymers or copolymers of the present invention that provide a bacteriostatic property to them. They can optionally contain suitable solvent(s), carrier(s), thickeners, pigments, fragrances, deodorizers, emulsifiers, surfactants, wetting agents, waxes, or oils. For example, in some aspects of the invention, the copolymers are incorporated into a formulation for external use as a pharmaceutically acceptable skin cleanser, particularly for the surfaces of human hands. Cleansers, polishes, paints, sprays, soaps, lotions, handwashes, and detergents are the like containing the polymers orcopolymers of the present invention are useful in homes and institutions, particularly but not exclusively in hospital settings for the prevention of nosocomial infections.

In other aspects of the invention, the polymers and copolymers of the invention are useful as preservatives and can be used in a method for killing or inhibiting the growth of a microbial species in a product. For example, the polymers and copolymers of the invention can be used as preservatives in cosmetics.

The polymers and copolymers also can be added to foodstuffs as a preservative. Foodstuffs that can be treated with polymers or copolymers of the invention include, but are not limited to, non-acidic foods, such as mayonnaise or other egg products, potato products, and other vegetable or meat products. The polymers and copolymers for adding to the foodstuff can be part of any comestible formulation that can also include a suitable medium or carrier for convenient mixing or dissolving into a particular foodstuff. The medium or carrier is preferably one that will not interfere with the familiar flavor of the food of interest, such as are known by the artisan skilled in food processing techniques.

In yet other aspects of the invention, the polymers and copolymers of the present invention provide a surface-mediated microbicide that only kills organisms in contact with the surface and are useful as surface-mediated disinfectants or preservatives.

Any object that is exposed to or susceptible to bacterial or microbial contamination can be treated with the copolymers of the present invention to provide a microbial surface. To provide a microbial surface, polymers and copolymers of the present invention are attached to, applied on or incorporated into almost any substrate including but not limited to woods, paper, synthetic polymers (plastics), natural and synthetic fibers, natural and synthetic rubbers, cloth, dry wall, glasses and ceramics by appropriate methods including covalent bonding, ionic interaction, coulombic interaction, hydrogen bonding or cross-linking. Examples of synthetic polymers include elastically deformable polymers which may be thermosetting or thermoplastic including, but not limited to polypropylene, polyethylene, polyvinyl chloride, polyethylene terephthalate, polyurethane, polyesters, such as polylactide, polyglycolide, rubbers such as polyisoprene, polybutadiene or latex, polytetrafluoroethylene, polysulfone and polyethylenesulfone polymers or copolymers. Examples of natural fibers include cotton, wool and linen.

The incidence of infection from food-borne pathogens is a continuing concern and antimicrobial packaging material, utensils and surfaces would be valuable. In the health care and medical device areas the utility of antimicrobial instruments, packaging and surfaces are obvious. Products used internally or externally in humans or animal health including, but not limited to, surgical gloves, implanted devices, sutures, catheters, dialysis membranes, water filters and implements, all can harbor and transmit pathogens.

Copolymers and polymers of the present invention are incorporated into any of these devices or implements to provide surface-medicated antimicrobial surfaces that will kill or inhibit the growth of organisms in contact with the surface. For example, polymers and copolymers of the present invention can be incorporated into spinnable fibers for use in materials susceptible to bacterial contamination including, but not limited to, fabrics, surgical gowns, and carpets. Also, ophthalmic solutions and contact lenses easily become contaminated and cause ocular infections. Antimicrobial storage containers for contact lens and cleaning solutions incorporating polymers and copolymers of the present invention would thus be very valuable.

Thus, in some embodiments, the present invention is directed to a method of killing or inhibiting the growth of a microorganism, the method comprising contacting the microorganism with an effective amount of a copolymer described above, for example, a random copolymer, as defined above, or a random copolymer having a monomer unit as defined above.

The polymers and copolymers of the present invention are synthesized using free-radical polymerization in the presence of a chain transfer agent. Standard methods of free radical polymerization are known to those of skill in the art. (See, for example, Mayo, F. R., J. Am. Chem. Soc. 65:2324-2329 (1943). See also “Polymer Synthesis: Theory and Practice” Third edition, D. Braun, H. Cherdron, H. Ritter, Springer-Verlag Berlin Heidelberg New York; Sanda, F., et al., Journal of Polymer Science: Part A: Polymer Chemistry, Vol. 36, 1981-1986 (1998); Henríquez, C., et al., Polymer 44:5559-5561 (2003); and De La Fuente, J. L., and Madruga, E. L., Journal of Polymer Science: Part A: Polymer Chemistry, Vol. 38, 170-178 (2000). See also Example 1 below, which provides a method for the synthesis of polynorbornene random copolymers.) For example, the polymers and copolymers of the present invention are synthesized by direct polymerization of two monomers, each containing a C—C double bond to produce polymers and copolymers.

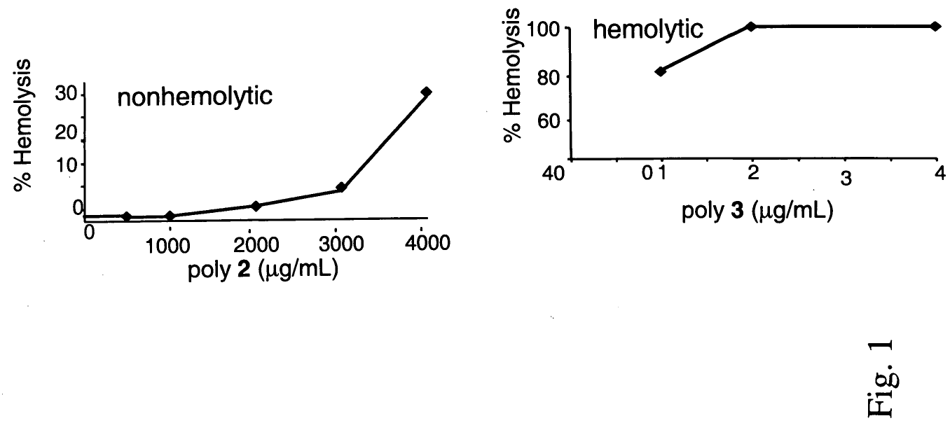

A general scheme illustrating free-radical polymerization of a polymer, as shown in Scheme 1 below, in the presence of a chain transfer agent is illustrated in FIG. 1A.

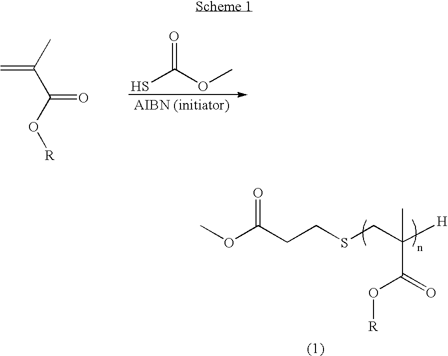

Where appropriate, a protecting group can be added to a side chain group of a monomer to protect the side chain during radical polymerization. For example, the tert-butoxycarbonyl (“BOC”) protecting group may be used to protect the free amine group of the monomer 2-aminoethyl methacrylate hydrochloride. Methods for chemically protecting reactive groups are known to those of skill in the art. See, for example, “Protective Groups in Organic Synthesis” Third edition, T. W. Greene, P. G. M. Wuts, John Wiley & Sons, Inc. (1999); and, for a description of radical polymerization of monomers having Boc protective groups, Sanda, F., et al., Journal of Polymer Science: Part A: Polymer Chemistry, Vol. 36, 1981-1986 (1998). Exemplary methods of synthesis is also described in Application Serial No. ______ entitled “Antimicrobial Copolymers and Uses Thereof” filed on Jul. 23, 2005, the entire contents of which are incorporated herein by reference. See also Example 1.

Monomers used in the synthesis of the copolymers of the present invention can be obtained commercially or prepared by methods known to those of skill in the art.

The polymers and copolymers of the present invention can be tested for antimicrobial activity by methods well known to those of skill in the art. See, for example, Tew, G. N., et al. (Tew, G. N., et al., Proc. Natl. Acad. Sci. USA 99:5110-5114 (2002)). Antimicrobial testing can be carried out using the micro-broth dilution technique with E. coli, or, if desired, another bacterial strain, such as, for example, B. subtilis, P. aeruginosa, K. pneumoniae, S. typhimurium, N. gonorrhoeae, B. megaterium, S. aureus, E. feacalis, M. luteus, or S. pyogenes. Other specific bacterial strains that can be screened include ampicillin and streptomycin-resistant E. coli D31, vancomycin-resistant Enterococcus faecium A436, and methicillin-resistant S. aureus 5332. Any polymer or copolymer found to be active can be purified to homogeneity and re-tested to obtain an accurate IC₅₀. Secondary screens include Klebsiella pneumoniae Kpl, and Salmonella typhimurium S5, and Pseudomonus aeruginosa 10. Traditionally, the micro-broth dilution technique only evaluates a single data point between 18-24 hours; however, the measurements can be extended to 24 hr to monitor cell growth through the entire growth phase. These experiments are performed in LB medium (which is a rich medium typically used to grow cells for protein expression) and represent a critical initial screen for activity. Since salt concentration, proteins, and other solutes can affect the activities of antibiotics, materials that show no activity in rich medium can be re-tested in minimal medium (M9) to determine if rich medium is limiting activity. No relationship between the media and the activity has been observed which is consistent with the mode of action this is believed to be through general membrane disruption.

Standard assays can be performed to determine whether a polymer or copolymer of the present invention is bacteriostatic or bactericidal. Such assays are well known to those of skill in the art and are performed, for example, by incubating E. coli cells overnight with the polymer or copolymer being tested, and then plating the mixture on agar plates according to procedures well known to those of skill in the art. See, for example, Tew, G. N., et al. (Tew, G. N., et al., Proc. Natl. Acad. Sci. USA 99:5110-5114 (2002)), and Liu, D., and DeGrado, W. F. (Liu, D., and DeGrado, W. F., J. Amer. Chem. Soc. 123:7553-7559 (2001)).

Assays for determining the antiviral and antifungal activity of polymers and copolymers of the present invention are also well known to those of skill in the art. For examples of antiviral assays, see Belaid et al., (Belaid, A., et al., J. Med. Virol. 66:229-234 (2002)), Egal et al., (Egal, M., et al., Int. J. Antimicrob. Agents 13:57-60 (1999)), Andersen et al., (Andersen, J. H., et al., Antiviral Rs. 51:141-149 (2001)), and Bastian, A., and Schafer, H. (Bastian, A., and Schafer, H., Regul. Pept. 15:157-161 (2001)). See also Cole, A. M., et al., Proc. Natl. Acad. Sci USA 99:1813-1818 (2002). For examples of antifungal assays, see Edwards, J. R., et al., Antimicrobial Agents Chemotherapy 33:215-222 (1989), and Broekaert, W. F., et al., FEMS Microbiol. Lett. 69:55-60 (1990). The entire contents of each of these documents is fully incorporated herein by reference.

Assays for measuring the cytotoxic selectivity for polymers and copolymers of the present invention toward bacteria and eukaryotic cells are well known to those of skill in the art. For example, cytotoxic selectivity can be assessed by determining the hemolytic activity of the polymers and copolymers. Hemolytic activity assays are performed by measuring the degree of hemolysis of human erythrocytes following incubation in the presence of the polymer and determining HC₅₀ values. HC₅₀ values represent the concentration of compound that results in 50% hemoglobin release. See, for example, Kuroda, K, and DeGrado, W. F., J. Amer. Chem. Soc. 127:4128-4129 (2005) and Liu, D., and DeGrado, W. F., J. Amer. Chem. Soc. 123:7553-7559 (2001), and references cited therein. See also Javadpour, M. M., et al., J. Med. Chem. 39:3107-3113 (1996).

Vesicle leakage assays can also be used to confirm whether a polymer of the present invention interacts with and disrupt phospholipid bilayers, a model for cellular membranes. Vesicle leakage assays are well known to those of skill, in the art. See, for example, Tew, G. N., et al. (Tew, G. N., et al., Proc. Natl. Acad. Sci. USA 99:5110-5114 (2002)), and references cited therein.

Assays for determining the heparin-neutralizing activity of polymers and copolymers of the present invention are well known to those of skill in the art and are commonly performed using either an activated partial thromboplastin time assay (for example, by measuring the delay in clotting times for activated plasma in the presence of a fixed concentration of heparin, in the absence and presence of a test compound) or a Factor X assay. See, for example, Kandrotas (Kandrotas, R. J., Clin. Pharmacokinet. 22:359-374 (1992)), Wakefield et al. (Wakefield, T. W., et al., J. Surg. Res. 63:280-286 (1996)), and Diness, V., and Østergaard, P. B. (Diness, V. O., and Østergaard, P. B., Thromb. Haemost. 56:318-322 (1986)), and references cited therein. See also Wong, P. C., et al., J. Pharm. Exp. Therap. 292:351-357 (2000), and Ryn-McKenna, J. V., et al., Thromb. Haemost. 63:271-274 (1990).

The polymers and copolymers of the present invention can be used to kill or inhibit the growth of any of the following microbes or mixtures of the following microbes, or, alternatively, can be administered to treat local and/or systemic microbial infections or illnesses caused by the following microbes or mixtures of the following microbes: Gram-positive cocci, for example Staphylococci (Staph. aureus, Staph. epidennidis) and Streptococci (Strept. agalactiae, Strept. faecalis, Strept. pneumoniae, Strept. pyogenes); Gram-negative cocci (Neisseria gonorrhoeae and Yersinia pestis) and Gram-negative rods such as Enterobacteriaceae, for example Escherichia coli, Hamophilus influenzae, Citrobacter (Citrob. freundii, Citrob. divernis), Salmonella and Shigella, and Francisella (Francisella tularensis); Gram-positive rods such as Bacillus (Bacillus anthracis, Bacillus thuringenesis); furthermore Klebsiella (Klebs. pneumoniae, Klebs. oxytoca), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. Furthermore, the antimicrobial spectrum of the copolymers of the present invention covers the genus Pseudomonas (Ps. aeruginosa, Ps. maltophilia) and strictly anaerobic bacteria such as, for example, Bacteroides fragilis, representatives of the genus Peptococcus, Peptostreptococcus and the genus Clostridium; furthermore Mycoplasmas (M. pneumoniae, M. hominis, Ureaplasma urealyticum) as well as Mycobacteria, for example Mycobacterium tuberculosis. This list of microbes is purely illustrative and is in no way to be interpreted as restrictive.

Examples of microbial infections or illness that can be treated by administration of the polymers and copolymers of the present invention include, but are not limited to, microbial infections or illnesses in humans such as, for example, otitis, pharyngitis, pneumonia, peritonitis, periodontal disease, pyelonephritis, cystitis, endocarditis, systemic infections, bronchitis (acute and chronic), septic infections, illnesses of the upper airways, diffuse panbronchiolitis, pulmonary emphysema, dysentery, enteritis, liver abscesses, urethritis, prostatitis, epididymitis, gastrointestinal infections, bone and joint infections, cystic fibrosis, skin infections, postoperative wound infections, abscesses, phlegmon, wound infections, infected burns, burns, infections in the mouth, infections after dental operations, osteomyelitis, septic arthritis, cholecystitis, peritonitis with appendicitis, cholangitis, intraabdominal abscesses, pancreatitis, sinusitis, mastoiditis, mastitis, tonsileitis, typhoid, meningitis and infections of the nervous system, salpingitis, endometritis, genital infections, pelveoperitonitis and eye infections.

Examples of viral infections that can be treated by administration of the polymers and copolymers of the present invention include, but are not limited to, viral infections caused by human immunodeficiency virus (HIV-1, HIV-2), hepatitis virus (e.g., hepatitis A, hepatitis B, hepatitis C, hepatitis D, and hepatitis E viruses), herpesviruses (e.g., herpes simplex virus types 1 and 2, varicella-zoster virus, cytomegalovirus, Epstein Barr virus, and human herpes viruses types 6, 7, and 8), influenza virus, respiratory syncytial virus (RSV), vaccinia virus, and adenoviruses. This list is purely illustrative and is in no way to be interpreted as restrictive.

Examples of fungal infections or illnesses that can be treated by administration of the polymers and copolymers of the present invention include, but are not limited to, fungal infections caused by Chytridiomycetes, Hyphochrytridiomycetes, Plasmodiophoromycetes, Oomycetes, Zygomycetes, Ascomycetes, and Basidiomycetes. Fungal infections which can be inhibited or treated with compositions of the copolymers provided herein include, but are not limited to: Candidiasis, including, but not limited to, onchomycosis, chronic mucocutaneous candidiasis, oral candidiasis, epiglottistis, esophagitis, gastrointestinal infections, genitourinary infections, for example, caused by any Candida species, including, but not limited to, Candida albicans, Candida tropicalis, Candida (Torulopsis) glabrata, Candida parapsilosis, Candida lusitaneae, Candida rugosa and Candida pseudotropicalis; Aspergillosis, including, but not limited to, granulocytopenia caused, for example, by, Aspergillus spp. including, but not limited, to Aspergillus fumigatus, Aspergillus favus, Aspergillus niger and Aspergillus terreus; Zygomycosis, including, but not limited to, pulmonary, sinus and rhinocerebral infections caused by, for example, zygomycetes such as Mucor, Rhizopus spp., Absidia, Rhizomucor, Cunningamella, Saksenaea, Basidobolus and Conidobolus; Cryptococcosis, including, but not limited, to infections of the central nervous system, e.g., meningitis, and infections of the respiratory tract caused by, for example, Cryptococcus neoformans; Trichosporonosis caused by, for example, Trichosporon beigelii; Pseudallescheriasis caused by, for example, Pseudallescheria boydii; Fusarium infection caused by, for example, Fusarium such as Fusarium solani, Fusarium moniliforme and Fusarium proliferartum; and other infections such as those caused by, for example, Penicillium spp. (generalized subcutaneous abscesses), Trichophyton spp., for example, Trichophyton mentagrophytes and Trichophyton rubrum, Stachybotrys spp., for example, S. chartarum, Drechslera, Bipolaris, Exserohilum spp., Paecilomyces lilacinum, Exophila jeanselmei (cutaneous nodules), Malassezia furfur (folliculitis), Alternaria (cutaneous nodular lesions), Aureobasidium pullulans (splenic and disseminated infection), Rhodotorula spp. (disseminated infection), Chaetomium spp. (empyema), Torulopsis candida (fungemia), Curvularia spp. (nasopharnygeal infection), Cunninghamella spp. (pneumonia), H. Capsulatum, B. dermatitidis, Coccidioides immitis, Sporothrix schenckii and Paracoccidioides brasiliensis, Geotrichum candidum (disseminated infection). The polymers and copolymers of the present invention can also be used to kill or inhibit the growth of any of the fungi listed above. This list is purely illustrative and is in no way to be interpreted as restrictive.

The polymers and copolymers of the present invention can be administered to a human subject. Thus, in some aspects of the invention, the polymers and copolymers are administered to a human.

The methods disclosed above also have veterinary applications and can be used to treat a wide variety of non-human vertebrates. Thus, in other aspects of the invention, the polymers and copolymers of the present invention are administered in the above methods to non-human vertebrates, such as wild, domestic, or farm animals, including, but not limited to, cattle, sheep, goats, pigs, dogs, cats, and poultry such as chicken, turkeys, quail, pigeons, ornamental birds and the like.

The following are examples of microbial infections in non-human vertebrates that can be treated by administering a polymer or copolymer of the present invention: Pig: coli diarrhoea, enterotoxaemia, sepsis, dysentery, salmonellosis, metritis-mastitis-agalactiae syndrome, mastitis; ruminants (cattle, sheep, goat): diarrhea, sepsis, bronchopneumonia, salmonellosis, pasteurellosis, mycoplasmosis, genital infections; horse: bronchopneumonias, joint ill, puerperal and post-puerperal infections, salmonellosis; dog and cat: bronchopneumonia, diarrhoea, dermatitis, otitis, urinary tract infections, prostatitis; poultry (chicken, turkey, quail, pigeon, ornamental birds and others): mycoplasmosis, E. coli infections, chronic respiratory tract illnesses, salmonellosis, pasteurellosis, psittacosis. This list is purely illustrative and is in no way to be interpreted as restrictive.

For those applications in which the polymers and copolymers of the present invention are used as disinfectants and/or preservatives, e.g., in cleansers, polishers, paints, sprays, soaps, or detergents, the polymers and copolymers are incorporated into the cleanser, polisher, paint, spray, soap, or detergent formulation, optionally in combination with suitable solvent(s), carrier(s), thickeners, pigments, fragrances, deodorizers, emulsifiers, surfactants, wetting agents, waxes, or oils. If the polymer or copolymer is to be used as a preservative in a foodstuff, it can be added to the foodstuff as part of any comestible formulation that can also include a suitable medium or carrier for convenient mixing or dissolving into the foodstuff. The amount added to or incorporated into the cleanser, polisher, soap, etc. formulation or into the foodstuff will be an amount sufficient to kill or inhibit the growth of the desired microbial species and can easily be determined by one of skill in the art.

For those applications in which the polymers and copolymers of the invention are used as surface-mediated microbicides, e.g., in some applications as disinfectants and as preservatives (e.g., including, but not limited to, medical devices such as catheters, bandages, and implanted devices, or food containers and food handling implements), the polymers and copolymers can be attached to, applied on or incorporated into almost any substrate including, but not limited to, woods, paper, synthetic polymers (plastics), natural and synthetic fibers, natural and synthetic rubbers, cloth, dry wall, glasses and ceramics by appropriate methods, including covalent bonding, ionic interaction, coulombic interaction, hydrogen bonding or cross-linking.

Procedures for attaching, applying, and incorporating the polymers and copolymers of the present invention into appropriate materials and substrates are disclosed in WIPO Publ. No. WO 02/100295, the contents of which are fully incorporated herein by reference. Appropriate substrates and materials are also disclosed in WO 02/100295.

The polymers and copolymers of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the polymers and copolymers of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication (e.g., whether the copolymers are administered to treat a microbial infection, or to provide an antidote for hemorrhagic conditions associated with heparin therapy). The mode of administration can depend on the pathogen or microbe to be targeted. The selection of the specific route of administration and the dose regimen is to be adjusted or titrated by the clinician according to methods known to the clinician in order to obtain the optimal clinical response. The amount of copolymer to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

For example, another embodiment of the present invention provides a composition of a polymer or copolymer of the present invention suitable for the treatment or prevention of oral diseases and a method of treating oral diseases by administering a random copolymer. Compositions suitable for treating oral diseases include, but are not limited to, pastes, gels, gums, topical liquids, sprays, inhalants or implantable devices for release into the oral tissue.

Pharmaceutical formulations containing the polymers and copolymers of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The polymers and copolymers of the present invention can be formulated for parenteral administration by injection, e.g., by bolus injection or continuous infusion. The copolymers can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the polymers and copolymers can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the polymers and copolymer compositions can take the form of, e.g., tablets or lozenges formulated in a conventional manner.

For administration by inhalation, the polymers and copolymers for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The polymers and copolymers of the present invention can also be formulated in rectal compositions such as suppositories or retention enemas, e.g., containing conventional suppository bases such as cocoa butter or other glycerides.

In addition to the formulations described previously, the polymers and copolymers of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the polymers and copolymers of the present invention, for example, can be applied to a plaster, or can be applied by transdermal, therapeutic systems that are consequently supplied to the organism.

Pharmaceutical compositions of the polymers and copolymers also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The polymers and copolymers of the present invention can also be administered in combination with other active ingredients, such as, for example, adjuvants, protease inhibitors, or other compatible drugs or compounds where such combination is seen to be desirable or advantageous in achieving the desired effects of the methods described herein (e.g., controlling infection caused by harmful microorganisms, or treating hemorrhagic complications associated with heparin therapy). For example, the polymers and copolymers of the present invention can be administered with other antibiotics, including, but not limited to, vancomycin, ciprofloxacin, merapenem, oxicillin, and amikacin.

The following examples will serve to further typify the nature of this invention but should not be construed as a limitation in the scope thereof, which scope is defined solely by the appended claims.

### EXAMPLE 1

Materials. (Tricyclohexylphosphine) (1,3-dimesitylimidazolidine-2-ylidine) benzylidineruthenium dichloride, the second generation Grubbs' catalyst, was purchased from Strem Chemical. Stearoyl-oleoyl-phosphatidylcholine (SOPC) and phosphatidylserine (SOPS) were purchased from Avanti Polar-Lipids, Inc. Cyclopentadiene for the synthesis of fulvene derivatives was obtained by the thermally induced cracking of dicyclopentadiene followed by distillation. Compounds 1-3, homopolymers of 1-4, and [(H₂Imes)(3-Br-py)₂-(Cl)₂Ru═CHPh] were prepared according to literature procedures. All other reagents were obtained from Aldrich. Deuterated chloroform and dichloromethane were passed through columns of basic activated alumina prior to use.

Instrumentation. ¹H (300 MHz), and ¹³C NMR (75 MHz) spectra were obtained on a Bruker DPX-300 NMR spectrometer. Gel permeation chromatography (GPC) was performed with a Polymer Lab LC1120 high-performance liquid chromatography (HPLC) pump equipped with a Waters differential refractometer detector. The mobile phase was tetrahydrofuran (THF) with a flow rate of 1.0 mL/min and 0.5 mL/min respectively. Separations were performed with 10⁵, 10⁴, and 10³ Å Polymer Lab columns. Molecular weights were calibrated versus narrow molecular weight polystyrene standards. Fluorescence spectroscopy was recorded with a Perkin Elmer LS50B Luminescence Spectrometer. Optical density and absorbance spectroscopy were recorded with a Molecular Devices SpectraMAX 190 plate reader.

Preparation of Compound 4. Compound 4 was prepared by a slight modification of the literature procedure that was used for the preparation of compounds 2 and 3. To a solution of 4-heptanone (20 mmol, 2.28 g) and cyclopentadiene (20 mmol, 1.32 g) in methanol (20 mL) was added pyrrolidine (20 mmol, 1.42 g). The mixture was stirred at room temperature for 1 hour and acetic acid was added (20.1 mmol, 1.21 g). The reaction mixture was diluted with ether (50 mL) and water (50 mL). Ether portion was separated, washed with water (50 mL) and brine (50 mL), and dried over MgSO₄. Ether was removed under reduced pressure and the product, di-n-propylfulvene, was used without further purification for the cycloaddition with maleic anhydride. The Diels-Alder reaction between di-n-propylfulvene (20 mmol, 3.24 g) and maleic anhydride (20 mmol, 1.96 g) was performed in ethyl acetate (50 mL) at 80° C. for 2 hours in a sealed pressure tube. Upon removal of ethyl acetate under reduced pressure, the adduct was obtained in high yield as an oil (85:15 exo-endo ratio) and used without further purification. Previously reported mono protected diamine (6.8 g, 42.3 mmol) was added to the Diels-Alder adduct (6.1 g, 23.5 mmol) in DMAc (N,N-Dimethylacetamide, 6 mL) at 60° C. and stirred for 20 minutes. A catalytic amount of cobalt acetate (0.5 mmol, 88.5 mg) dissolved in DMAc was added to this mixture followed by the addition of acetic anhydride (25 mmol, 255 mg) and the reaction mixture was stirred for 4 hours at 80° C. After cooling to room temperature the solution was diluted with ethyl acetate, washed with water and dilute HCl, dried, and evaporated under reduced pressure to afford 95% yield of an exo-endo (87:13) mixture of Compound 4. Recrystallization from cold diethyl ether afforded pure exo isomer 4 (50%). ¹H NMR (300 MHz, CDCl₃, ppm): δ 6.42 (2H, t, J=2.1 Hz), 5.05 (1H, s), 3.70 (2H, t, J=1.9 Hz), 3.53 (2H, t, J=5.4 Hz), 3.25 (2H, broad d, J=5.0 Hz), 2.75 (2H, s), 1.82 (4H, t, J=7.8 Hz), 1.42 (9H, s), 1.22 (4H, m), 0.81 (6H, t, J=7.3 Hz). ¹³C NMR (75 MHz, CDCl₃, ppm): δ 177.6, 155.8, 141.9, 137.8, 123.2, 78.9, 47.8, 45.1, 38.8, 38.4, 33.1, 28.2, 21.7, 13.9. HRMS (FAB) calcd for C₂₃H₃₅N₂O₄: 403.260. Found: 403.260.

Preparation of poly4. Homopolymerizations of compound 4 and subsequent deprotection of primary amine groups to obtain poly4 were performed according to the previously reported literature procedure, using bromo pyridine substituted derivative of second generation Grubbs' catalyst, [(H₂Imes)(3-Br-py)₂— (Cl)₂Ru═CHPh].^{45 1}H NMR (300 MHz, D₂O, ppm): δ 5.70-5.20 (2H, br), 4.10-3.50 (4H, br), 3.40-3.05 (4H, br), 2.20-1.70 (4H, br), 1.55-1.10 (4H, br), 1.00-0.60 (6H, s). ¹³C NMR (75 MHz, d-DMSO, ppm): δ 178.6 (br), 138.1 (br), 135.8, 132.4 (br), 51.3 (br), 47.9 (br), 44.2, 36.2, 33.5, 21.0, 13.8.

Preparation of random copolymers. The preparation of poly(2₂-co-3₁) (Mn=15300 g/mol) is described as a representative procedure for the preparation of random copolymers of compound 2 and compound 3. Comonomer feed ratio and catalyst to monomer ratio were changed in order to obtain random copolymers with desired comonomer content and molecular weights. A mixture of 2 (0.58 mmol) and 3 (0.29 mmol) was dissolved in dichloromethane (1.5 mL) and a solution of catalyst (0.015 mmol in 0.05 mL of dichloromethane), [(H₂Imes)(3-Br-py)₂—(Cl)₂Ru═CHPh], was added at room temperature, under an inert atmosphere. The mixture was allowed to react for 90 minutes at 40° C. Polymerization was terminated by addition of ethyl vinyl ether (0.2 mL) followed by precipitation in pentane resulting in a white polymer precipitate and brown supernatant. The product was filtered and dried overnight under reduced pressure at room temperature. A small sample was used for molecular weight determination. Deprotection of primary amine pendant groups was performed by dissolution of the polymer in trifluoroacetic acid and stirring at 45° C. for 8 hours. Polymer was recovered by evaporation of trifluoroacetic acid under reduced pressure and dissolution in water followed by freeze-drying overnight. The isolated yield was 85% (275 mg). ¹H NMR (300 MHz, D₂O, ppm): δ 5.90-5.10 (2H, br), 4.35-3.55 (4H, br), 3.55-2.90 (4H, br), 2.65-2.30 (33% of 1H, br), 2.00-1.20 (66% of 6H, br), 1.10-0.60 (33% of 6H, br). ¹³C NMR (75 MHz, D20, ppm): δ 180.4 (br), 163.7, 163.4, 163.2, 162.8, 162.3, 139.4 (br), 136.0 (br), 134.9 (br), 132. 2 (br), 131.4 (br), 130.6 (br), 122.6, 118.7, 114.9, 111.0, 52.8, 51.6 (br), 50.0 (br), 48.5 (br), 46.4 (br), 37.8, 36.7, 28.8 (br), 22.5, 21.0.

Measurement of hemolytic activity. Hemolytic activity measurements were performed with slight modifications of literature procedures.^{7,12,47} Freshly drawn human red blood cells (HRBC, 30 μL), were suspended in 10 mL TRIS saline (10 mM TRIS, 150 mM NaCl, pH 7.2, filtered through polyethersulfone membrane with 0.20 μm pore size) and rinsed 3 times by centrifugation (5 minutes at 1500 rpm) and resuspension in TRIS saline. Polymer solutions were prepared by dissolution in TRIS saline (10 mM TRIS, 150 mM NaCl, pH 7.2) at concentration of 8 mg/mL and further diluted as necessary. After the complete dissolution the pH of the solution was adjusted to pH values between about 6.5 and about 7.0 depending on the solubility of polymer. TRIS saline solutions of poly1, poly2, and poly(2-co-3) were adjusted to about pH 7.0. TRIS saline solutions of poly3, and poly4 were adjusted to about pH 6.5 because of slow precipitation of these polymers at higher pH values. After the pH adjustments, polymer solutions were filtered through polyethersulfone membranes (0.45 μm pore size). Freshly prepared polymer solutions with different concentrations were added to 100 μL of the above-prepared HRBC suspension to reach a final volume of 200 μL on a 96-well plate. The resulting mixture was kept at about 37° C. for about 30 minutes on a stirring plate. Then the plate was centrifuged (10 minutes at 1500 rpm) and the supernatant in each well was transferred to a new plate. Hemolysis was monitored by measuring the absorbance of the released hemoglobin at 414 nm. 100% hemolysis was obtained by adding 1% TRITON-X, a strong surfactant, to the above-prepared HRBC suspension. The upper limit of polymer concentration that was required to cause 50% hemolysis is reported as HC₅₀, where the absorbance from TRIS saline containing no polymer was used as 0% hemolysis. The value of percent hemolysis was reported in cases where it was below 50% hemolysis at the highest polymer concentration tested or above 50% hemolysis at the lowest polymer concentration tested. Relatively small absorbance of polymer solution due to residual catalyst at 414 nm, at the corresponding concentrations, were measured and subtracted from polymer-HRBC mixtures. All experiments were run in quadruplicate. Control experiments were run in order to monitor the hemolytic activity of TFA treated ruthenium catalyst that may be present in trace amounts in polymer solutions. Catalyst was dissolved and stirred for 8 hours at 45° C. in TFA followed by evaporation of TFA and dissolution in DMSO due to the insolubility of TFA treated catalyst in TRIS saline. No hemolytic activity was observed from the catalyst solution within the time and concentration limits that were used for the hemolysis studies.

Measurement of antibacterial activity. Antibacterial activity measurements were performed with slight modifications of literature procedures. Bacteria suspension (E. coli D31 and B. subtilus ATCC 8037), which was grown in Muller-Hinton Broth (MHB) overnight at 37° C., diluted with fresh MHB to an optical density of 0.1 at 600 nm (OD₆₀₀) and further diluted by a factor of 10. This suspension was mixed with different concentrations of freshly prepared polymer solutions in TRIS saline (pH 6.5-7.0) in a 96-well plate and incubated for 6 hours at 37° C. The OD₆₀₀ was measured for bacteria suspensions that were incubated in the presence of polymer solution or only TRIS saline. Antibacterial activity was expressed as minimal inhibitory concentration (MIC), the concentration at which 90% inhibition of growth was observed after 8 hours. All experiments were run in quadruplicate. In a control experiment, the TFA treated ruthenium catalyst did not show any antibacterial activity within the time and concentration limits that were used for antibacterial activity assays.

Determination of polymer-induced leakage of vesicle content. The lipid vesicles were prepared with slight modifications of literature procedures. Cholesterol (1.7 μmol) was dissolved in a chloroform solution of SOPC (17.2 μmol) and the chloroform was subsequently removed under a nitrogen stream followed by drying under reduced pressure for 3 hours at room temperature to obtain the mixture as a dry film. The dried film was hydrated by addition of 2 mL of buffer containing calcein (40 mM) and sodium phosphate (10 mM, pH 7.0). The suspension was vortexed for 10 min. The suspension was sonicated three times in a bath type sonicator (Aquasonic 150 HT) at room temperature and freeze-thawed after each sonication. The non-encapsulated calcein was removed by eluting through a size exclusion Sephadex G-25-150 column with 90 mM sodium chloride, 10 mM sodium phosphate buffer (pH 7) as eluent. The preparation of negatively charged SOPS/SOPC vesicles and the measurement of polymer-induced calcein leakage from lipid vesicles were performed according to a literature procedure.

Results and Discussion.

Amphiphilic polynorbornene derivatives. The biological activities of a class of amphiphilic polymers that were previously shown to exhibit lipid membrane disruption activities was tested. The amphiphilic polynorbornene derivatives bearing primary amine and variable length alkyl moieties as pendant groups were prepared by ROMP of modular norbornene derivatives using the [(H₂Imes)(3-Brpy)₂-(Cl)₂Ru═CHPh] variant of Grubbs' catalyst. These amphiphilic polymers provide a well-defined model for testing the effect of hydrophobicity and molecular weight of cationic polymers on antibacterial and hemolytic activities. The current study involves four types of repeating units (1-4) as below. 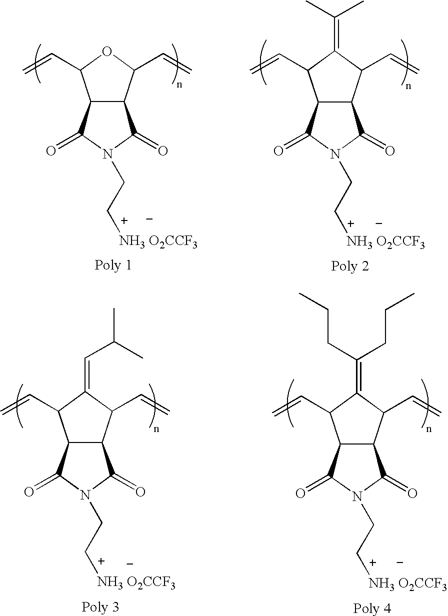
All homo and copolymers of these monomers have narrow polydispersities, less than about 1.3, and encompass a large range of molecular weight from oligomers to high polymers, up to about 137500 g/mol. No preformed and stable polymeric secondary structure is expected from these macromolecules considering the imperfect tacticity of polynorbornene derivatives prepared by homogeneous ruthenium catalyst, and the presence of cis-trans isomers on the backbone unsaturations. Furthermore, the asymmetry in the isobutylidene group of poly3 results from head-to-head and head-to-tail insertions leads to multiple dyad possibilities. In the case of random copolymers, there is the factor of additional compositional heterogeneity. All polymers are soluble in TRIS saline solutions at appropriate pH values (about 6.5-7.0).

Antibacterial and hemolytic activities of homopolymers. The hydrophobicity of the repeating unit was observed to effect antibacterial and hemolytic activities of the amphiphilic polymers. The activity of each homopolymers with similar molecular weights (near 10,000 g/mol, Mₙ) was probed against Gram-negative bacteria (E. coli), Gram-positive bacteria (B. subtilus), and human red blood cells. Results are depicted in Table 1 (Table 1).

TABLE 1
Antibacterial and hemolytic activities of homopolymers

| | MIC | | | Selectivity | |
|---|---|---|---|---|---|
| Poly- | [μg/mL, (μM)] | | HC₅₀ | (HC₅₀/MIC) | |
| mer | E. coli | B. subtilus | [μg/mL, (μM)]^{†} | E. coli | B. subtilus |
| Poly1 | >500, (>49) | >500, (>49) | >1000, (>98) | — | — |
| Poly2 | 200, (20) | 300, (30) | >4000, (>400) | >20 | >13 |
| Poly3 | 25, (2.5) | 25, (2.5) | <1, (<0.1) | <0.04 | <0.04 |
| Poly4 | 200, (19) | 200, (19) | <1, (<0.1) | <0.005 | <0.005 |

Mₙ and PDI values are 10250 g/mol, 1.07 for poly1, 9950 g/mol, 1.10 for poly2, 10050 g/mol, 1.13 for poly3, and 10300 g/mol, 1.08 for poly4. Mn and PDI values were determined by THF GPC relative to polystyrene standards, prior to deprotection of the polymer.^{†} Poly1 caused 5% hemolysis at 1000 μg/mL, the highest concentration measured. Poly2 caused 25% hemolysis at 4000 μg/mL. Poly3 caused 80% hemolysis at 1 μg/mL, and poly4 caused 100% hemolysis at 1 μg/mL, the lowest concentrations measured.

Poly1, a cationic polymer with no substantial hydrophobic group, did not show any observable antibacterial or hemolytic activity within the measured concentrations. This result is consistent with the previously reported lack of activity against phospholipid membranes. Introduction of a hydrophobic group at the repeat unit level produced an increase in antibacterial and hemolytic activities, which appeared to depend on the size of hydrophobic group. Poly2, with an isopropylidene pendant group, exhibited antibacterial activity with MIC of 200 μg/mL against E. coli, which is less efficacious than most antimicrobial peptides, and their mimics, that have MICs typically ranging between 1-50 μg/mL. However, poly2 remained non-hemolytic up to the measured concentration of 4000 μg/mL, thus giving a selectivity, defined as the ratio of HC to MIC, greater than about 20. Poly3, with an additional carbon atom per repeat unit, appears to be more hydrophobic than poly2, and has additional mobility of the pendant alkyl group. Poly3 exhibited substantial increase in antibacterial activity, with MIC of 25 μg/mL for both E. coli and B. subtilus as well as hemolytic activity, HC₅₀ less than 1 μg/mL (Table 1). This increase in antibacterial and hemolytic activity with increasing hydrophobicity is in accordance with literature reports that predict larger hydrophobic groups will have stronger interactions with the inner core of cell membranes leading to loss of selectivity. However, in the case of poly4, when the hydrophobic size was further increased the hemolytic activity was retained, but the antibacterial activity decreased to a MIC of 200 μg/mL. In many instances, hydrophobic interactions have been reported to control hemolytic activities; whereas charge interactions are suggested to be more important for antibacterial activity. These results indicate that the presence, and balance, of hydrophobic and hydrophilic groups dictate the antibacterial and hemolytic activities of the amphiphilic non-natural polymer in agreement with natural peptide studies.

The effect of molecular weight on antibacterial and hemolytic activities was investigated for poly2, poly3, and poly4. Results are show in Table 2.

TABLE 2
Effect of molecular weight on antibacterial
and hemolytic activities

| | | | MIC | | | |
|---|---|---|---|---|---|---|
| | Mₙ | | [μg/mL, (μM)] | | HC₅₀ | |
| Polymer | (g/mol) | PDI | E. coli | B. subtilus | [μg/mL, (μM)]^{†} | |
| Poly2 | 1600 | 1.15 | 200, (125) | 300, (188) | >4000, | (>2500) |
| | 24100 | 1.10 | 200, (8.3) | 200, (8.3) | >4000, | (>164) |
| | 49600 | 1.14 | 200, (4.0) | 200, (4.0) | >4000, | (>81) |
| | 137500 | 1.27 | 200, (1.5) | 200, (1.5) | >4000, | (>29) |
| Poly3 | 1650 | 1.26 | 25, (15) | 25, (15) | <1, | (<0.6) |
| | 25500 | 1.17 | 40, (1.6) | 40, (1.6) | <1, | (<0.04) |
| | 57200 | 1.70 | 80, (1.4) | 80, (1.4) | <1, | (<0.02) |
| Poly4 | 5300 | 1.09 | 200, (38) | 200, (38) | <1, | (<0.2) |
| | 32200 | 1.13 | 200, (6.2) | 200, (6.2) | <1, | (<0.04) |
| | 57000 | 1.19 | 200, (3.5) | 200, (3.5) | <1, | (<0.02) |

Mₙ and PDI values were determined by THF GPC relative to polystyrene standards, prior to the deprotection of polymer. ^{†}Poly2s caused 20-25% hemolysis at 4000 μg/mL. Poly3s caused 70-80% hemolysis at 1 μg/mL. Poly4s caused 100% hemolysis at 1 μg/mL.

Changes in molecular weights over a large range did not result in significant changes in antibacterial and hemolytic activities of poly2 and poly4. The antibacterial activity of poly3 was observed to increase moderately as the molecular weight decreased from 57200 g/mol to 10300 g/mol or lower. Overall there was no substantial molecular weight dependence on antibacterial or hemolytic activities of these homopolymers, if activity is reported in mass/volume rather than molarity. In the most commonly suggested mechanisms for membrane disruption based on amphiphilic peptides, there is some type of cooperative action, either in pore formation or coverage of the surface in a carpet-like manner. If the membrane disruption activity is associated with the accumulation of the macromolecule on the membrane surface, it is a germane approach to report MIC values in units of mass/volume. Otherwise at the same molar concentrations higher molecular weight polymers would cover larger surfaces than lower molecular weight polymers. However, it should be noted that this approach underestimates the possible effect of the increase in the number of electrostatic and hydrophobic interactions at the membrane surface as a consequence of covalent connectivity resulting from higher molecular weights. One of many possible advantages of high molecular weight polymeric systems would be the ability of using them at relatively low molar concentrations if that is a requirement of the target application.

Antibacterial and hemolytic activities of random copolymers. The results from homopolymerization studies have shown the strong influence of subtle structural changes on the biological activities of these amphiphilic polymers. The low hemolytic activity of poly2 and strong antibacterial activity of poly3 suggests that copolymerization of monomers 2 and 3 would be a facile synthetic approach to optimize activity and selectivity. Random copolymers consisting of different comonomer ratios of 2 and 3 were prepared without compromising narrow polydispersities. Random progression of the copolymerizations was confirmed by performing in situ ¹H NMR analysis. The synthetic approach employed allows various compositions to be explored in contrast to polycondensation approaches earlier reported. Poly(2₉-co-3₁), the random copolymer of 2 and 3 with a final comonomer molar ratio of 9/1 respectively and Mₙ of 12000 g/mol, showed antibacterial activity near that of poly3 while retaining the non-hemolytic character of poly2 as shown in Table 3.

TABLE 3
Activities of random copolymers of 2 and 3

| | | | MIC [μg/mL, (μM)] | | | Selectivity (HC₅₀/MIC) | |
|---|---|---|---|---|---|---|---|
| Polymer | Mₙ(g/mol) | PDI | E. coli | B. subtilus | HC₅₀ [μg/mL, (μM)]^{†} | E. coli | B. subtilus |
| Poly(2₉-co-3₁) | 12000 | 1.09 | 40, (3.3) | 40, (3.3) | >4000, (>333) | >100 | >100 |
| Poly(2₂-co-3₁) | 15300 | 1.15 | 40, (2.6) | 40, (2.6) | >4000, (>261) | >100 | >100 |
| | 93700 | 1.21 | 80, (0.9) | 80, (0.9) | >4000, (>43) | >50 | >50 |
| Poly(2₁-co-3₂) | 8500 | 1.09 | 40, (4.7) | 40, (4.7) | <1, (<0.12) | <0.025 | <0.025 |
| | 32600 | 1.19 | 80, (2.5) | 80, (2.5) | <1, (<0.03) | <0.013 | <0.013 |
| Poly(2₁-co-3₄) | 11800 | 1.15 | 40, (3.4) | 40, (3.4) | <1, (0.08) | <0.025 | <0.025 |

Mₙ and PDI values were determined by THF GPC relative to polystyrene standards, prior to the deprotection of polymer. ^{†}Poly(2₉-co-3₁) caused 15% hemolysis and poly(2₂-co-3₁)s caused 20-25% hemolysis at 4000 μg/mL. Poly(2₁-co-3₂)s caused 60-70% hemolysis and poly(2₁-co-3₄) caused 75% hemolysis at 1 μg/mL.

Notably, about 10% of comonomer 3 content was enough to bring the antibacterial activity near homopolymers of 3 and exhibit excellent selectivity, a ratio greater than 100. Poly(2₂-co-3₁)s have also shown high selectivity where antibacterial activity was slightly decreased with increasing molecular weight as in the case of poly3. These copolymers, with selectivity values reaching over 100, are powerful examples of the ability to obtain good antibacterial activity from non-hemolytic polymers by fine-tuning the hydrophobic/hydrophilic balance and molecular weight. Poly(2₁-co-3₂)s and poly(2₁-co-3₄)s exhibited high hemolytic activities in accordance with the increased content of hemolytic comonomer 3.

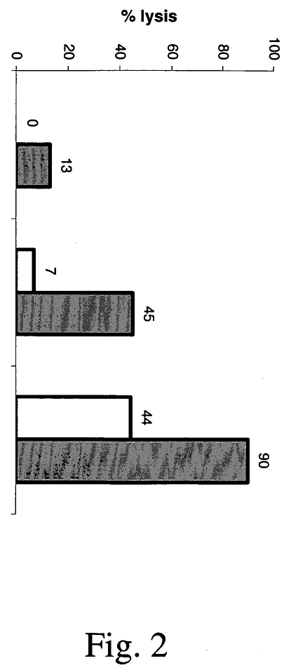

Disruption of lipid vesicle membranes. Polymer induced fluorescent dye leakage from negatively charged and neutral large unilamellar vesicles (LUV) were measured. Lipid vesicles provide simplified models for bacterial and mammalian cell membranes although they underestimate several factors such as cell walls and lipopolysaccharides in bacterial cell membranes. At the same time, these assays are well documented in the literature and provide useful insight. Therefore, these tests were used to study the overall membrane disruption activities of polymers but not to make direct comparisons of the activities against vesicles or biological cells. As shown in FIG. 2, Poly2 did not appear to be active against neutral vesicles and showed little disruption of negatively charged vesicles at the measured concentrations. Poly(2₂-co-3₁)s were found to exhibit increased activity against negatively charged vesicles while retaining low activities against neutral vesicles, with a selectivity near 6. Poly3 was highly active against both types of membranes with a lower selectivity of 2. Oligomers of poly3, with molecular weights ranging between 1,500 and 2,000 g/mol (Mn), have no significant activity on vesicles despite their high antibacterial and hemolytic activities (not shown). The above results confirm the membrane activity of these biologically active high molecular weight polymers but underestimates the degree of selectivity measured for poly(2₂-co-3₁)s during in vitro experiments. FIG. 2 depicts the percent lysis of neutral vesicles (cholesterol/SOPC) and negatively charged vesicles (SOPS/SOPC) of poly2, poly (2₂-co-3₁) and poly3.

Conclusion. Amphiphilic polymers based on modular norbornene derivatives were shown to exhibit good antibacterial activities and high selectivity for bacteria versus red blood cells. This class of polymers was prepared through a ROMP-based facile synthetic strategy that allows excellent control over monomer composition, molecular weight, polydispersity, and amphiphilicity. Small modifications to the hydrophobic character of the cationic amphiphilic polymer were shown to dramatically change the antibacterial and hemolytic activities. Tuning the hydrophobic/hydrophilic balance and molecular weights of these copolymers allowed preparation of highly selective, antibacterial non-hemolytic macromolecules. Desired biological activities were maintained across a large range of molecular weights. Furthermore, this study showed the preparation of fully synthetic high molecular weight polymers that mimic the activities of host-defense peptides in the absence of a specific secondary structure.

### EXAMPLE 2

This example illustrates example amphiphilic copolymers of the present invention, wherein the copolymers comprise a hydrophilic polynorbornene monomeric unit and a hydrophobic polynorbornene monomeric unit. Diels-Alder chemistry using furan and malaimde produced the bicyclic NH compound which is further reacted with either a nonpolar or polar group using standard methods. These methods can include either alkylation under basic conditions or alkylation using mitsunobu conditions. The primary amine groups are protected using standard protecting groups. For the basic alkylation, halides are used as the leaving group. For mitsunobu conditions, alcohols are employed.

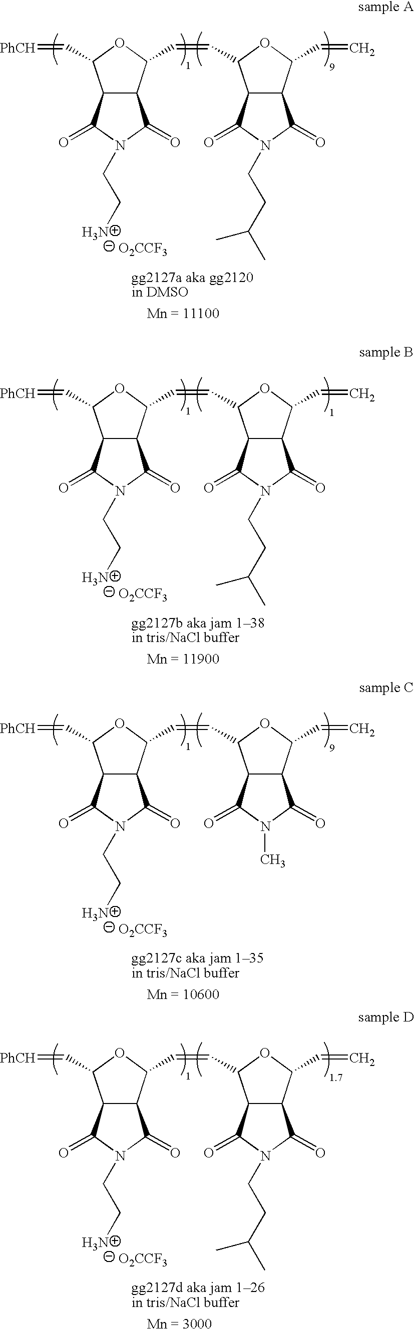

### EXAMPLE 3

This example illustrates the antimicrobial action of amphiphilic polynorbornene polymers of the present invention in various mechanical applications. Poly3 was incorporated into water-based formulations of paint and polyurethane and polyvinyl chloride. Specifically, polyurethane (PU) samples were prepared by mixing the appropriate amount of active polymer (poly3) in DMSO with 1 mL of PU. PVC was prepared by dissolving in tetrahydrofuran (THF) and mixing identical amounts as for PU. For painted surfaces, the active polymer was added to the paint as a solution or as a dry powder. These were then coated onto glass slides and allowed to dry overnight. The surfaces were sterilized with ethanol and then sprayed with bacteria. The bacteria were allowed to stand on the surface for various times between 3-30 minutes. It was then rinsed and collected with PBS, diluted appropriately, and spread onto agar plates. These plates were allowed to grow overnight and the colonies counted. Results are depicted in Tables 4 and 5 below.

TABLE 4
Antimicrobial action of polynorbornene polymer (Poly3) in paint

| | |
|---|---|
| Paint | Poly3 |

| Formulation | Control | 0.5% | 1% |
|---|---|---|---|
| WaterbaseA | >270 | 31 | 113 |
| WaterbaseB | >300 | >300 | 4 |
| WaterbaseC | >300 | >300 | 15 |

TABLE 5
Antimicrobial action of polynorbornene polymer (poly3) in plastic

| | | | |
|---|---|---|---|
| Material | Untreated | DMSO | Poly3 |

Polyurethane

| | | | |
|---|---|---|---|
| Trial 1 | 261 | >300 | 0 |
| Trial 2 | >300 | >300 | 0 |

Polyvinyl chloride

| | | | |
|---|---|---|---|
| Trial 1 | >300 | >300 | 2 |
| Trial 2 | >300 | >300 | 0 |
| Trial 3 | >400 | >200 | 2 |

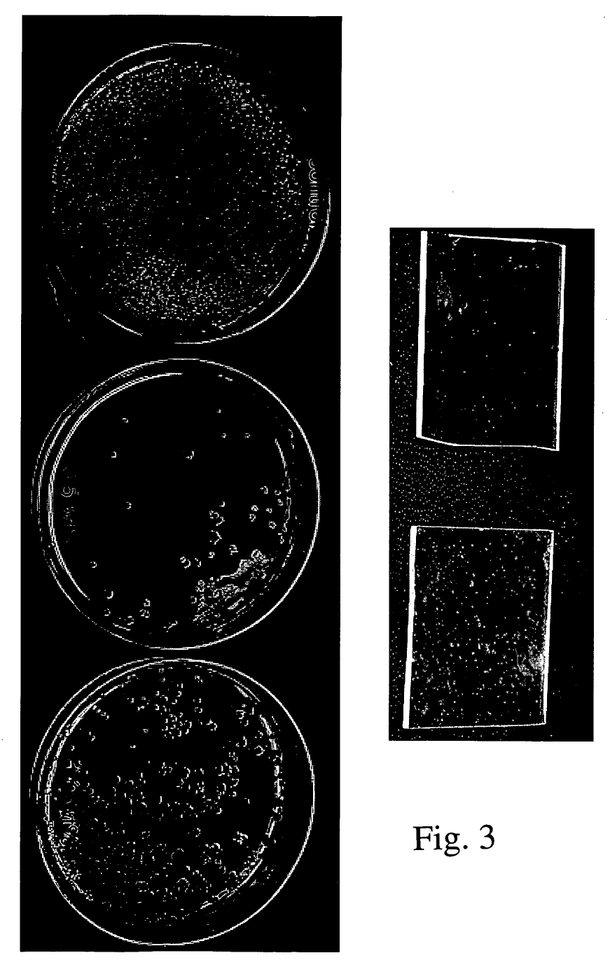

In another example, as depicted in FIG. 3, colony counts of commercial outdoor polyurethane paint containing about 0.5% weight of Poly3 (Fap) compared to control, containing no Poly 3 exhibited a significant decrease in living cells from the control. and shown in FIG. 3.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. A polynorbornene monomer comprising the formula 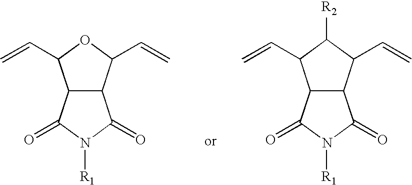
wherein R₁ is polar or non-polar and R₂, if present, is of the opposite polarity of R₁.

2. The monomer of claim 1, wherein said polynorbornene monomer is selected from the group consisting of

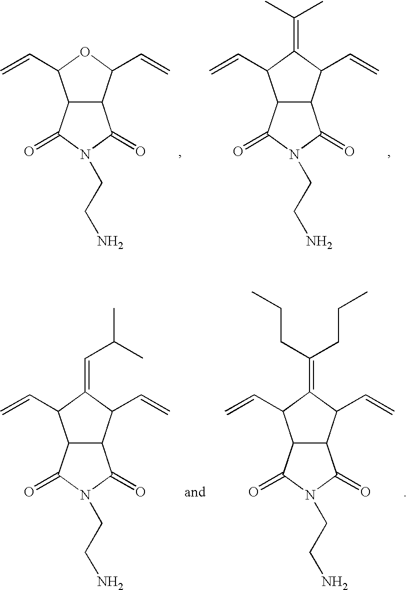

3. A polymer formed from a monomer of claim 1.

4. The polymer of claim 3, wherein said monomer is selected from the group consisting of 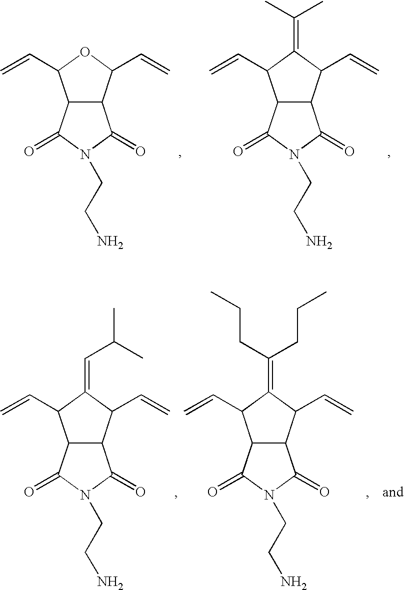
combinations thereof.

5. The polymer of claim 3 further comprising a second polynorbornene monomer.

6. The polymer of claim 5, wherein said polymer is block, random or alternating.

7. The polymer of claim 5, wherein said first amphiphilic monomer is poly2 and said second amphiphilic monomer is poly3.

8. The polymer of claim 7, wherein the ratio of poly2 to poly3 is about 10:1 to about 1:10.

9. The polymer of claim 7, wherein the ratio of poly2 to poly3 is about 1:1.

10. An amphiphilic monomer comprising the formula: 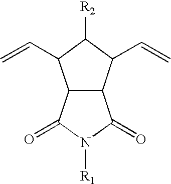
wherein R₁ is polar or non-polar and R₂ is of the opposite polarity of R₁.

11. The amphiphilic monomer of claim 10, wherein said polynorbornene monomer is selected from the group consisting of

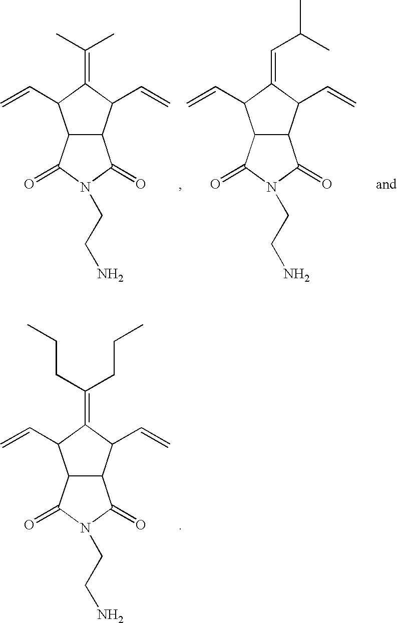

12. A polymer formed from a monomer of claim 10.

13. An amphiphilic copolymer comprising a polar polynorbornene monomeric unit and a non-polar polynorbornene monomeric unit.

14. The amphiphilic copolymer of claim 13, wherein said copolymer is block, random or alternating.

15. The amphiphilic copolymer of claim 13, wherein said ratio of polar to non-polar polynorbornene monomeric units is about 10:1 to about 1:10.

16. The amphiphilic copolymer of claim 13, wherein said ratio of polar to non-polar polynorbornene monomeric units is about 1:1.

17. The amphiphilic copolymer of claim 13, wherein said polar and non-polar polynorbornene monomeric units are selected from the group consisting of: 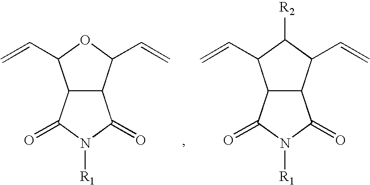
and combinations thereof wherein R₁ is polar or non-polar and R₂, if present, is of the same polarity of R₁.

18. The amphiphilic copolymer of claim 13, wherein said amphiphilic copolymer is selected from the group consisting of:

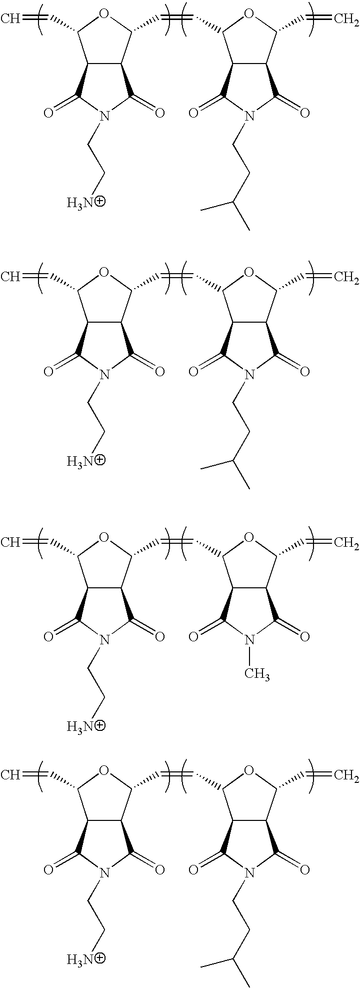

19. A pharmaceutical composition comprising the amphiphilic polymer of claim 3.

20. The pharmaceutical composition of claim 19, wherein said composition is administered topically, orally, or intravenously.

21. A pharmaceutical composition comprising the amphiphilic copolymer of claim 13.

22. The pharmaceutical composition of claim 21, wherein said composition is administered topically, orally, or intravenously

23. A method of treating a microbial infection comprising administering a therapeutically effective amount of the amphiphilic polymer of claim 3.

24. The method of claim 23, wherein said microbial infection is a bacterial infection, a fungal infection or a viral infection.

25. A method of treating a microbial infection comprising administering a therapeutically effective amount of the amphiphilic copolymer of claim 13.

26. The method of claim 25, wherein said microbial infection is a bacterial infection, a fungal infection or a viral infection.

27. A method of inhibiting the growth of a microorganism comprising administering an effective amount of the amphiphilic polymer of claim 3.

28. A method of inhibiting the growth of a microorganism comprising administering an effective amount of the amphiphilic copolymer of claim 13.

29. A method of inhibiting microbial growth on or in a material comprising applying the amphiphilic polymer of claim 3 to said material.

30. The method of claim 29, wherein said application step comprises coating said material.

31. The method of claim 29, wherein said application step comprises spraying said material.

32. The method f claim 29, wherein said application step comprises mixing said polymer with said material.

33. The method of claim 29, wherein said material is selected form the group paint, lacquer, coating, varnish, caulk, grout, adhesive, resin, film, cleanser, polish, cosmetic, soap, lotion, handwash, and detergent.

34. A method of inhibiting microbial growth on or in a material comprising applying the amphiphilic copolymer of claim 13 to said material.

35. The method of claim 34, wherein said application step comprises coating said material.

36. The method of claim 34, wherein said application step comprises spraying said material.

37. The method of claim 34, wherein said application step comprises mixing said polymer with said material.

38. The method of claim 34, wherein said material is selected form the group paint, lacquer, coating, varnish, caulk, grout, adhesive, resin, film, cleanser, polish, cosmetic, soap, lotion, handwash, and detergent.

39. An antimicrobial composition comprising at least one active and hemolytic polynorbornene monomer and at least one less active and less hemolytic polynorbornene monomer.

40. The antimicrobial composition of claim 36, wherein said active and hemolytic monomer comprises poly3.

41. The antimicrobial composition of claim 36, wherein said less active and less hemolytic monomer is poly2.

42. A method of preparing an amphiphilic polymer comprising polymerizing a more than one polynorbornene monomer having the formula selected from the group consisting 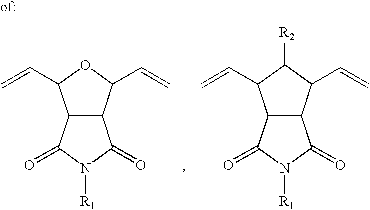
and a combination thereof.
